(19) United States
(12) Patent Application Publication
(10) Pub. No.: US 2016/0242435 A1
(43) Pub. Date: Aug. 25, 2016

(54) HIGH QUALITY PROTEIN CONCENTRATE FROM CANOLA MEAL

(71) Applicant: PRAIRIE AQUATECH, DAST LLC, BROOKINGS, SD (US)

(72) Inventors: WILLIAM GIBBONS, BROOKINGS, SD (US); JASON CROAT, BROOKINGS, SD (US)

(73) Assignee: PRAIRIE AQUATECH, DAST LLC, Brookings, SD (US)

(21) Appl. No.: 15/047,952

(22) Filed: Feb. 19, 2016

Related U.S. Application Data
(60) Provisional application No. 62/176,497, filed on Feb. 19, 2015.

(51) Int. Cl. A23K 10/12; A23K 20/10; A23K 10/30; C07K 14/415; A23K 10/37

(52) U.S. Cl. CPC A23K 10/12; C07K 14/415; A23K 10/37; A23K 10/30; A23K 20/10

(57) ABSTRACT

The present invention describes fungal conversion methods for reduction of glucosinolates (GLS), fiber and residual sugars to increase the protein content and nutritional value of canola meal, including canola-based protein products (CBPP) generated by said methods for use in animal feeds.

### CROSS REFERENCE TO RELATED APPLICATION

This application claims benefit under 35 U.S.C. §119(e) to U.S. Provisional Application No. 62/176,497, filed Feb. 19, 2015, which is incorporated by reference herein in its entirety.

This work was made with Governmental support from the United States Department of Agriculture under contract 2015-00505. The Government has certain rights in this invention.

### BACKGROUND OF THE INVENTION

1. Field of the Invention

The present invention relates generally to incubation processes, and specifically to microbial incubation processes to produce high quality protein concentrates, including products made thereform and use of such products in the formulation of nutrient feeds.

2. Background Information

Canola (Brassica napus) is grown widely in Canada and the northern U.S. as a source of edible oil or for biodiesel/j et fuel production. The U.S. Department of Agriculture's National Agricultural Statistics Service estimated the 2014 US canola crop at 2.52 billion pounds harvested over 1.55 million acres. Following oil extraction, the remaining meal is used as a protein source for livestock. On a worldwide basis, canola meal is second only to soybean meal for use as a feed. There has been much interest in utilizing rapeseed meal to replace soybean meal in ruminant and monogastric feeds. However, a limitation of meals from Brassica spp. is the presence of glucosinolates (GLS), which are anti-nutritional and can even be toxic at high ingestion levels.

GLS and the enzyme myrosinase are compartmentally stored separately in Brassica spp. Upon disruption of plant tissues, myrosinase cleaves glucose from GLS, which in turn is converted into toxic compounds such as nitriles, thiocyanates, and isothiocyanates. This self-defense mechanism evolved to reduce animal and insect browsing of the plant. When consumed, these toxic breakdown products can cause deleterious effects on the thyroid, and ultimately cause goiters from iodine deficiency. For this reason canola was bred to contain lower levels of GLS and erucic acid. However, feed inclusion rates are still limited to ˜30%, and this reduces the value of canola meal.

In 2006, MCN Bioproducts Inc. patented a mechanical process to concentrate and purify canola proteins (see, U.S. Pat. No. 7,629,041). This process is similar to methods currently used to produce soy protein concentrate and isolate, and achieves a product with up to 80% protein by weight dry matter. Unfortunately, the multiple separation steps of this process are expensive and result in a low protein yield, since proteins also fractionate into lower value co-products. The result is an expensive product similar to soy protein isolate that is more suitable for use in human foods.

Therefore, a process for generating quality plant-derived protein which is cost-effective and “green”, where the generated protein is of a high enough quality to use in the formulation of nutrient feeds, is needed.

### SUMMARY OF THE INVENTION

The present invention relates to fungal conversion culture to reduce glucosinolates (GLS), fiber and residual sugars to increase the protein content and nutritional value of canola meal, including the use of solid state and submerged incubation conditions and the generation of protein concentrates and canola-based protein products (CBPP) for use in animal feeds.

In embodiments, a method of producing a protein concentrate from canola using fermentation is disclosed including contacting canola containing substrate with a microbe selected from Trichoderma reesei or Fusarium venenatum; incubating the contacted substrate for sufficient time, and at a pH of between about 4.0 to about 6.0, to convert the carbohydrates contained in the substrate into protein to form a protein concentrate; and isolating the resulting protein concentrate, where the resulting protein concentrate exhibits a reduction in glucosinolate concentration of between about 69% to about 98%.

In one aspect, fermentation is solid-state fermentation (SSF) or submerged fermentation (SMF) or a hybrid of SSF and SMF. In another aspect, the microbe is Trichoderma reesei.

In one aspect, the fermentation is SSF. In a related aspect, the moisture content of the substrate is between about 40% to about 60%.

In another aspect, the protein concentration of the substrate increases between about 15% to about 23%.

In one aspect, the substrate is thermally treated prior to contact with the microbe. In another aspect, the substrate is hexane extracted (HE) or cold pressed (CP). In a further aspect, incubation is carried out without perturbing the substrate.

In a related aspect, a protein concentrate produced by the methods as described herein is disclosed.

In one embodiment, a method of producing a non-animal based protein concentrate is disclosed including pelletizing a substantially dry substrate selected from cereal grains, bran, sawdust, peat, oil-seed materials, wood chips, and combinations thereof; passing moisture through said pelletized substrate; inoculating the moisturized pellets by fogging atomized inoculant onto the moisturized pellets, where the inoculant comprises at least one microbe; incubating the inoculated pellets at a suitable temperature under solid state fermentation conditions in a first chamber; transferring the incubated pellets to one or more second chambers; drying and milling the transferred pellets; and recovering the resulting protein concentrate comprising the at least one microbe.

In one aspect, the at least one microbe includes Aureobasidium pullulans, Sclerotium glucanicum, Sphingomonas paucimobilis, Ralstonia eutropha, Rhodospirillum rubrum, Kluyveromyces and Pichia spp, Trichoderma reesei, Pleurotus ostreatus, Rhizopus spp, and combinations thereof.

In another aspect, the method further includes exposing said pellets to UV light prior to inoculating said moisturized pellets.

In one embodiment, a method of producing a protein concentrate from canola using fermentation is disclosed including contacting canola containing, liquid substrate with a microbe selected from Trichoderma reesei or Fusarium venenatum; incubating the contacted substrate for sufficient time, and at a pH of between about 4.0 to about 6.0, to convert the carbohydrates contained in the substrate into protein to form a protein concentrate, where incubation is carried out with perturbation of the substrate; and isolating the resulting protein concentrate, where the resulting protein concentrate exhibits a reduction in glucosinolate concentration of between about 69% to about 98%.

In one aspect, the canola substrate includes extruded canola meal. In a related aspect, the canola meal is hexane extracted (HE) or cold pressed (CP).

In another aspect, the incubation is carried out for about 168 hours. In a further aspect, the method includes drying the resulting isolated protein concentrate.

### BRIEF DESCRIPTION OF THE DRAWINGS

FIG. 1a shows surface colonization of various fungi on hexane-extracted (HE) canola meal.

FIG. 1b shows surface colonization of various fungi on cold-pressed (CP) canola meal.

FIG. 2a shows maximal protein and residual sugar levels of HE canola meal.

FIG. 2b shows maximal protein and residual sugar levels of CP canola meal.

FIG. 3a shows maximal protein levels±SD of HE canola meal following submerged fungal incubations.

FIG. 3b shows maximal protein levels±SD of CP canola meal following submerged fungal incubation.

FIG. 4a shows residual sugar levels±SD of HE canola meal following submerged fungal incubation.

FIG. 4b shows residual sugar levels±SD of CP canola meal following submerged fungal incubation.

FIG. 5a shows reduction of total GLS±SD following sterilization submerged fungal incubation in HE canola meal.

FIG. 5b shows reduction of total GLS±SD sterilization submerged fungal incubation in CP canola meal.

FIG. 6a shows maximal protein levels±SD in HE canola meal following submerged fungal incubation.

FIG. 6b shows maximal protein levels±SD in CP canola meal following submerged fungal incubation.

FIG. 7a shows reduction of total residual sugar levels±SD from raw HE canola meal by pretreatment and submerged fungal incubation.

FIG. 7b shows reduction of total residual sugar levels±SD from raw CP canola meal by pretreatment and submerged fungal incubation.

FIG. 8a shows NDF fiber levels±SD in HE canola meal following submerged fungal incubation.

FIG. 8b shows NDF fiber levels±SD in CP canola meal following submerged fungal incubation.

FIG. 9a shows reduction of total GLS levels±SD from raw HE canola meal by pretreatment and submerged fungal incubation.

FIG. 9b shows reduction of total GLS levels±SD from raw CP canola meal by pretreatment and submerged fungal incubation.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term. In embodiments, composition may “contain”, “comprise” or “consist essentially of” a particular component of group of components, where the skilled artisan would understand the latter to mean the scope of the claim is limited to the specified materials or steps “and those that do not materially affect the basic and novel characteristic(s)” of the claimed invention.

As used herein, the term “animal” means any organism belonging to the kingdom Animalia and includes, without limitation, humans, birds (e.g. poultry), mammals (e.g. cattle, swine, goal, sheep, cat, dog, mouse and horse) as well as aquaculture organisms such as fish (e.g. trout, salmon, perch), mollusks (e.g. clams) and crustaceans (e.g. lobster and shrimp).

Use of the term “fish” includes all vertebrate fish, which may be bony or cartilaginous fish.

As used herein “non-animal based protein” means that the protein concentrate comprises at least 0.81 g of crude fiber/100 g of composition (dry matter basis), which crude fiber is chiefly cellulose and lignin material obtained as a residue in the chemical analysis of vegetable substances.

As used herein, “incubation process” means the provision of proper conditions for growth and development of bacteria or cells, where such bacteria or cells use biosynthetic pathways to metabolize various feedstocks. In embodiments, the incubation process may be carried out, for example, under aerobic conditions. In other embodiments, the incubation process may include fermentation.

As used herein, the term “incubation products” means any residual substances directly resulting from an incubation process/reaction. In some instances, an incubation product contains microorganisms such that it has a nutritional content enhanced as compared to an incubation product that is deficient in such microorganisms. The incubation products may contain suitable constituent(s) from an incubation broth. For example, the incubation products may include dissolved and/or suspended constituents from an incubation broth. The suspended constituents may include undissolved soluble constituents (e.g., where the solution is supersaturated with one or more components) and/or insoluble materials present in the incubation broth. The incubation products may include substantially all of the dry solids present at the end of an incubation (e.g., by spray drying an incubation broth and the biomass produced by the incubation) or may include a portion thereof. The incubation products may include crude material from incubation where a microorganism may be fractionated and/or partially purified to increase the nutrient content of the material.

As used herein, “perturbed” means to disturb or physically agitate (e.g., mix or blend).

As used herein, a “conversion culture” means a culture of microorganisms which are contained in a medium that comprises material sufficient for the growth of the microorganisms, e.g., water and nutrients. The term “nutrient” means any substance with nutritional value. It can be part of an animal feed or food supplement for an animal. Exemplary nutrients include but are not limited to proteins, peptides, fats, fatty acids, lipids, water and fat soluble vitamins, essential amino acids, carbohydrates, sterols, enzymes and trace minerals, such as, phosphorus, iron, copper, zinc, manganese, magnesium, cobalt, iodine, selenium, molybdenum, nickel, fluorine, vanadium, tin, and silicon.

Conversion is the process of culturing microorganisms in a conversion culture under conditions suitable to convert protein/carbohydrate/polysaccharide materials, for example, soybean material into a high-quality protein concentrate. Adequate conversion means utilization of 90% or more of specified carbohydrates to produce microbial cell mass and/or exopolysaccharide. In embodiments, conversion may be aerobic or anaerobic.

As used herein a “flocculent” or “clearing agent” is a chemical that promotes colloids to come out of suspension through aggregation, and includes, but is not limited to, a multivalent ion and polymer. In embodiments, such a flocculent/clearing agent may include bioflocculents such as exopolysaccharides.

As used herein, “solid state fermentation” (SSF) refers to a process of combining a liquid inoculum comprising microbe/cells along with a solid to achieve a mixture having a moisture level of about 40% to about 60%. In embodiments, the SSF is run for 5 to 7 days.

To generate a less expensive canola protein concentrate, the instant methods take advantage of the metabolic diversity of fungi to convert canola fiber and carbohydrates into protein-rich cell biomass, while simultaneously degrading GLS and the breakdown products. In embodiments, processes are disclosed to generate a more digestible product with enhanced nutritional value to a range of aquaculture and other livestock species. Based on their ability to produce cellulose degrading enzymes, fungi may include Aureobasidium pullulans, Trichoderma reesei, Fusarium venenatum, Pichia kudriavzevii, and Mucor circinelloides. In one aspect, cold pressed and hexane extracted canola meals may serve as substrates for incubation.

In embodiments, single-step, solid-state incubation process is disclosed, as it replicates the natural environment to which fungi are adapted. In a related aspect, fungal mycelia can effectively penetrate solid substrate agro-industrial residues. In one aspect, solid state conditions also limit bacterial contamination due to the reduced water activity. Lower drying costs and the ability to use smaller incubation vessels, compared to submerged incubation processing, help minimize industrial processing costs. In embodiments, SSF reduces raffinose to between about 0.00 to about 1.9%, and reduces stachyose to between about 0.00 to about 2.1%. In a related aspect, SSF reduces GLS from about 60.6 to about 1.0 μM/g, or about 50.7 to about 65.5%. In a further related aspect, SSF increases protein concentration about 15.4% to about 22.9%. Unless otherwise denoted, all values are in w/w %.

As disclosed herein, solid-state incubation with various fungal strains has proved beneficial to enhance the nutritional composition of canola meal. In embodiments, T. reesei (NRRL-3653), A. pullulans (NRRL-58522), and A. pullulans (Y-2311-1) resulted in the greatest improvement in protein content, exhibiting maximum protein increases of 22.9, 16.9 and 15.4%, respectively, in solid-state incubated cold pressed canola meal. These strains also reduced total glucosinolate content to the greatest extent.

As disclosed herein, fungal cultures may be used to reduce glucosinolates, fiber, and residual sugars while increasing the protein content and nutritional value of canola meal. As demonstrated herein, solid-state incubation conditions were used to enhance filamentous growth of the fungi. In one aspect, flask trials were performed using 50% moisture content hexane extracted or cold pressed canola meal, with incubation for 168 h at 30° C. On hexane extracted canola meal Trichoderma reesei (NRRL-3653) achieved the greatest increase in protein content (23%), while having the lowest residual levels of sugar (8% w/w) and glucosinolates (0.4 mg/g). On cold pressed canola meal Trichoderma reesei (NRRL-3653), A. pullulans (NRRL-58522), and A. pullulans (NRRL-Y-2311-1) resulted in the greatest improvement in protein content (22.9, 16.9 and 15.4%, respectively), while reducing total glucosinolate content to 1.0, 1.2 and 4.3 mg/g, respectively.

In embodiments, submerged incubation (SmF) conditions were used to evaluate performance of seven fungal cultures in hexane extracted (HE) and cold pressed (CP) canola meal. In one aspect, Aureobasidium pullulans (Y-2311-1), Fusarium venenatum and Trichoderma reesei resulted in the greatest improvement in protein levels in HE canola meal, at 21.0, 23.8 and 34.8%, respectively. These fungi reduced total GLS content to 2.7, 7.4, and 4.9 uM/g, respectively, while residual sugar levels ranged from 0.8 to 1.6% w/w. In another aspect, for trials with CP canola meal, the same three fungi increased proteins levels by 24.6, 35.2, and 37.3%, and final GLS levels to 6.5, 4.0 and 4.7 uM/g respectively. Additionally, residual sugar levels were reduced to 0.3 to 1.0% w/w. In embodiments, SmF reduces raffinose to between about 0.37 to about 2.4%, and reduces stachyose to between about 0.00 to about 2.1%.

In one embodiment, submerged incubation (SmF) conditions were used to evaluate four pretreatment methods (extrusion, hot water cook, dilute acid, and dilute alkali) and three fungal cultures (Aureobasidium pullulans (Y-2311-1), Fusarium venenatum NRRL-26139, and Trichoderma reesei NRRL-3653) in hexane extracted (HE) and cold pressed (CP) canola meal. In one aspect, the combination of extrusion pretreatment followed by incubation with T. reesei resulted in the greatest overall improvement to HE canola meal, increasing protein to 51.5%, which reducing NDF (neutral detergent fiber), GLS and residual sugars to 18.6%, 17.2 uM/g, and 5% w/w, respectively. In another aspect, extrusion pretreatment and incubation with F. venenatum performed the best with CP canola meal resulting in 54.4% protein while reducing NDF, GLS, and residual sugars to 11.6%, 6.7 uM/g and 3.8 w/w, respectively. In embodiments, depending on the pretreatment, SmF reduces raffinose to between about 0.00 to about 3.7%, and reduces stachyose to between about 0.00 to about 2.8%.

The main reason for using plant proteins in the feed industry is to replace more expensive protein sources, like animal protein sources. Another important factor is the danger of transmitting diseases through feeding animal proteins to animals of the same or related species. Examples for plant protein sources include, but are not limited to, the plant family Brassiciaceae as exemplified by canola. These protein sources, also commonly defined as oilseed proteins may be fed whole, but they are more commonly fed as a by-product after oils have been removed.

In the fish farming industry the major fishmeal replacers with plant origin reportedly used, include, but are not limited to, soybean meal (SBM), maize gluten meal, Rapeseed/canola (Brassica sp.) meal, lupin (Lupinus sp. like the proteins in kernel meals of de-hulled white (Lupinus albus), sweet (L. angustifolius) and yellow (L. luteus) lupins, Sunflower (Helianthus annuus) seed meal, crystalline amino acids; as well as pea meal (Pisum sativum), Cottonseed (Gossypium sp.) meal, Peanut (groundnut; Arachis hypogaea) meal and oilcake, soybean protein concentrate, corn (Zea mays) gluten meal and wheat (Triticum aestivum) gluten, Potato (Solanum tuberosum L.) protein concentrate as well as other plant feedstuffs like Moringa (Moringa oleifera Lam.) leaves, all in various concentrations and combinations.

The protein sources may be in the form of non-treated plant materials and treated and/or extracted plant proteins.

A protein material includes any type of protein or peptide. In embodiments, canola material or the like may be used such as whole rapeseeds. Whole rapeseeds may be standard, commoditized rapeseeds; rapeseeds that have been genetically modified (GM) in some manner; or non-GM identity preserved rapeseeds. Exemplary GM rapeseeds include, for example, rapeseed engineered to resist pathogens, resistant to pests, enriched for amino acids, vitamins, specific fatty acids, and the like. In addition, fractionated rapeseed meal may also be used (e.g., fine or course as denoted by air classification).

Other types of rapeseed material include canola protein flour, canola protein concentrate, canola meal and canola protein isolate, or mixtures thereof. The traditional processing of whole rapeseeds into other forms of canola protein such as canola protein flours, canola protein concentrates, canola meal and canola protein isolates, includes crushing the seed and defatting in a known manner, as by extraction with an organic solvent, followed by removal of the solvent. This process removes all or most of the oil, and leaves a material known as oilseed flake. Optionally, this flake may be toasted, and the product of such toasting is oilseed meal. Both flake and meal are defatted oilseeds within the scope of the instant invention. Flake and meal are rich in proteins. Canola protein has a good balance of essential amino acids, is of low molecular weight and is not highly allergenic to humans or animals.

The protein sources may be in the form of non-treated plant materials and treated and/or extracted plant proteins. As an example, heat treated soy products have high protein digestibility. Still, the upper inclusion level for full fat or defatted soy meal inclusion in diets for carnivorous fish is between an inclusion level of 20 to 30%, even if heat labile antinutrients are eliminated. In fish, soybean protein has shown that feeding fish with protein concentration inclusion levels over 30% causes intestinal damage and in general reduces growth performance in different fish species. In fact, most fish farmers are reluctant to use more than 10% plant proteins in the total diet due to these effects.

The present invention solves this problem and allows for plant protein inclusion levels of up to 40 or even 50%, depending on, amongst other factors, the animal species being fed, the origin of the plant protein source, the ratio of different plant protein sources, the protein concentration and the amount, origin, molecular structure and concentration of the glucan and/or mannan. In embodiments, the plant protein inclusion levels are up to 40%, preferably up to 20 or 30%. Typically the plant protein present in the diet is between 5 and 40%, preferably between 10 or 15 and 30%. These percentages define the percentage amount of a total plant protein source in the animal feed or diet, this includes fat, ashes etc. In embodiments, pure protein levels are up to 50%, typically up to 45%, in embodiments 5-95%.

The proportion of plant protein to other protein in the total feed or diet may be 5:95 to 95:5, 15:85 to 50:50, or 25:75 to 45:55.

### Microorganisms

The disclosed microorganisms must be capable of converting carbohydrates and other nutrients into a high-quality protein concentrate in a conversion culture. In embodiments, the microorganism is a yeast-like fungus. An example of a yeast-like fungus is Aureobasidium pullulans. Other example microorganisms include yeast such as Kluyveromyces and Pichia spp, Lactic acid bacteria, Trichoderma reesei, Pleurotus ostreatus, Rhizopus spp, and many types of lignocellulose degrading microbes. Generally, exemplary microbes include those microbes that can metabolize stachyose, raffinose, xylose and other sugars. However, it is within the abilities of a skilled artisan to pick, without undue experimentation, other appropriate microorganisms based on the disclosed methods.

In embodiments, the microbial organisms that may be used in the present process include, but are not limited to, Aureobasidium pullulans, Trichoderma reesei, Fusarium venenatum, Pichia kudriavzevii and Mucor circinelloides, and combinations thereof.

### Conversion Culture

In exemplary embodiments, after pretreatment, the protein material (such as extruded canola meal) may be blended with water at a solid loading rate of at least 5%, with pH adjusted to 4.0-6.0. Then appropriate dosages of hydrolytic enzymes may be added and the slurry incubated with agitation at 150-250 rpm at 50° C. for 3-24 h. After cooling to 35° C., an inoculum of microbe may be added and the culture may be incubated for an additional 72-120 h, or until the carbohydrates are consumed. During incubation, sterile air may be sparged into the reactor at a rate of 0.5-1 L/L/h. In embodiments, the conversion culture undergoes conversion by incubation with the soybean material for less than about 96 hours. In embodiments, the conversion culture will be incubated for between about 96 hours and about 120 hours. In embodiments, the conversion culture may be incubated for more than about 120 hours. The conversion culture may be incubated at about 35° C.

In embodiments, solid state conditions include use of sterilized (e.g., via autoclave) canola meal substrates having between about 40-50% moisture content (HE or CP), where the pH is adjusted for each organism. The sterilized substrate is inoculated with the subject organism and incubated at about 30° C. for up to 168 h, where resulting solids are recovered.

In embodiments, the pH of the conversion culture, while undergoing conversion, may be about 4.0 to about 7.0. In embodiments, the conversion culture may be actively aerated.

The canola based protein product (CBPP), as well as pullulan, siderophores and/or other exopolysaccharides, may be recovered from the conversion culture following the conversion process by optionally alcohol precipitation and centrifugation. An example alcohol is ethanol, although the skilled artisan understands that other alcohols should work. In embodiments, salts may also be used to precipitate. Exemplary salts may be salts of potassium, sodium and magnesium chloride. In embodiments, a polymer or mutlivalent ions may be used alone or in combination with the alcohol. In embodiments, no flocculent or alcohol is necessary to isolate the CBPP.

In embodiments, final protein concentrations solids recovery may be modulated by varying incubation times. For example, about 75% protein may be achieved with a 14 day incubation, where the solids recovery is about 16-20%. In embodiments, incubation for 2-2.5 days increase solids recovery to about 60-64%, and protein level of 58-60% in the HQPC. In embodiments, 4-5 day incubation may maximize both protein content (e.g., but not limited to greater than about 70%) and solids recovery (e.g., but not limited to greater than about 60%). These numbers may greater or lower, depending on the feed stock. In embodiments, the protein concentrates (i.e., CBPP) may have a specific lipid:protein ratio, e.g., at about 0.010:1 to about 0.03:1, about 0.020:1 to about 0.025:1 or about 0.021:1 to about 0.023:1.

In embodiments, feed stocks may be extruded in a single screw extruder (e.g., BRABENDER PLASTI-CORDER EXTRUDER Model PL2000, Hackensack, N.J.) with a barrel length to screw diameter of 1:20 and a compression ratio of 3:1, although other geometries and ratios may be used. Feed stocks may be adjusted to about 10% to about 15% moisture, to about 15%, or to about 25% moisture. The temperature of feed, barrel, and outlet sections of extruder may be held at between about 40° C. to about 50° C. or to about 50° C. to about 100° C., about 100° C. to about 150° C., about 150° C. to about 170° C., and screw speed may be set at about 50 rpm to about 75 rpm or about 75 rpm to about 100 rpm or about 100 rpm to about 200 rpm to about 250 rpm. In embodiments, the screw speed is sufficient to provide a shearing effect against the ridged channels on both sides of a barrel. In embodiments, screw speed is selected to maximize sugar release.

In embodiments, extruded feed stock materials (e.g., plant proteins) may be mixed with water to achieve a solid loading rate of at least 5% in a reactor (e.g., a 5 L NEW BRUNSWICK BIOFLO 3 BIOREACTOR; 3-4 L working volume). The slurry may be autoclaved, cooled, and then saccharified by subjection to enzymatic hydrolysis using a cocktail of enzymes including, but not limited to, endo-xylanase and beta-xylosidase, Glycoside Hydrolase, α- or β-glucosidases, α- or β-galactosidases, hemicellulase activities. In one aspect, the cocktail of enzymes includes NOVOZYME® enzymes. Dosages to be may include 6% CELLICCTEK® (per gm glucan), 0.3% CELLICHTEK® (per gm total solids), and 0.15% NOVOZYME 960® (per gm total solids). Saccharification may be conducted for about 12 h to about 24 h at 40° to about 50° C. and about 150 rpm to about 200 rpm to solubilize the fibers and oligosaccharides into simple sugars. The temperature may then be reduced to between about 30° C. to about 37° C., in embodiments to about 35° C., and the slurry may be inoculated with 2% (v/v) of a 24 h culture of the microbe. The slurry may be aerated at 0.5 L/L/min and incubation may be continued until sugar utilization ceases or about 96 h to about 120 h. In fed-batch conversions more extruded feed stock may be added during either saccharification and/or the microbial conversion phase.

In embodiments, the cellulolytic composition may comprise one or more enzymes selected from an esterase, a cellulase, an expansin, a laccase, a ligniolytic enzyme, a pectinase, a peroxidase, a protease, a swollenin, or a phytase, where the enzymes may be added exogenously and/or produced through one or more microbes during incubation.

In embodiments, the feed stock and/or extrudate may be treated with one or more antibiotics (e.g., but not limited to, tetracycline, penicillin, erythromycin, tylosin, virginiamycin, and combinations thereof) before inoculation with the converting microbe to avoid, for example, contamination by unwanted bacteria strains.

During incubation, samples may be removed at 6-12 h intervals. Samples for HPLC analysis may be boiled, centrifuged, filtered (e.g., through 0.22-μm filters), placed into autosampler vials, and frozen until analysis. In embodiments, samples may be assayed for carbohydrates and organic solvents using a WATERS HPLC system, although other HPLC systems may be used. Samples may be subjected to plate or hemocytometer counts to assess microbial populations. Samples may also be assayed for levels of cellulose, hemicellulose, and pectin using National Renewable Energy Laboratory procedures.

### Dietary Formulations

In exemplary embodiments, the high-quality protein concentrate recovered from the conversion culture that has undergone conversion is used in dietary formulations. In embodiments, the recovered CBPP will be the primary protein source in the dietary formulation. Protein source percentages in dietary formulations are not meant to be limiting and may include 24 to 80% protein. In embodiments, the CBPP will be more than about 50%, more than about 60%, or more than about 70% of the total dietary formulation protein source. Recovered CBPP may replace protein sources such as fish meal, soybean meal, wheat and corn flours and glutens and concentrates, and animal byproduct such as blood, poultry, and feather meals. Dietary formulations using recovered CBPP may also include supplements such as mineral and vitamin premixes to satisfy remaining nutrient requirements as appropriate.

In certain embodiments, performance of the CBPP, may be measured by comparing the growth, feed conversion, protein efficiency, and survival of animal on a high-quality protein concentrate dietary formulation to animals fed control dietary formulations, such as fish-meal. In embodiments, test formulations contain consistent protein, lipid, and energy contents. For example, when the animal is a fish, viscera (fat deposition) and organ (liver and spleen) characteristics, dress-out percentage, and fillet proximate analysis, as well as intestinal histology (enteritis) may be measured to assess dietary response.

As is understood, individual dietary formulations containing the recovered CBPP may be optimized for different kinds of animals. In embodiments, the animals are fish grown in commercial aquaculture. Methods for optimization of dietary formulations are well-known and easily ascertainable by the skilled artisan without undue experimentation.

Complete grower diets may be formulated using CBPP in accordance with known nutrient requirements for various animal species. In embodiments, the formulation may be used for yellow perch (e.g., 42% protein, 8% lipid). In embodiments, the formulation may be used for rainbow trout (45% protein, 16% lipid). In embodiments, the formulation may be used for any one of the animals recited supra.

Basal mineral and vitamin premixes for plant-based diets may be used to ensure that micro-nutrient requirements will be met. Any supplements (as deemed necessary by analysis) may be evaluated by comparing to an identical formulation without supplementation; thus, the feeding trial may be done in a factorial design to account for supplementation effects. In embodiments, feeding trials may include a fish meal-based control diet. Pellets for feeding trials may be produced using the lab-scale single screw extruder (e.g., BRABENDER PLASTI-CORDER EXTRUDER Model PL2000).

### Feeding Trials

In embodiments, a replication of four experimental units per treatment (i.e., each experimental and control diet blend) may be used (e.g., about 60 to 120 days each). Trials may be carried out in 110-L circular tanks (20 fish/tank) connected in parallel to a closed-loop recirculation system driven by a centrifugal pump and consisting of a solids sump, and bioreactor, filters (100 μm bag, carbon and ultra-violet). Heat pumps may be used as required to maintain optimal temperatures for species-specific growth. Water quality (e.g., dissolved oxygen, pH, temperature, ammonia and nitrite) may be monitored in all systems.

In embodiments, experimental diets may be delivered according to fish size and split into two to five daily feedings. Growth performance may be determined by total mass measurements taken at one to four weeks (depending upon fish size and trial duration); rations may be adjusted in accordance with gains to allow satiation feeding and to reduce waste streams. Consumption may be assessed biweekly from collections of uneaten feed from individual tanks. Uneaten feed may be dried to a constant temperature, cooled, and weighed to estimate feed conversion efficiency. Protein and energy digestibilities may determined from fecal material manually stripped during the midpoint of each experiment or via necropsy from the lower intestinal tract at the conclusion of a feeding trial. Survival, weight gain, growth rate, health indices, feed conversion, protein and energy digestibilities, and protein efficiency may be compared among treatment groups. Proximate analysis of necropsied fishes may be carried out to compare composition of fillets among dietary treatments. Analysis of amino and fatty acids may be done as needed for fillet constituents, according to the feeding trial objective. Feeding trial responses of dietary treatments may be compared to a control (e.g., fish meal) diet response to ascertain whether performance of CBPP diets meet or exceed control responses.

Statistical analyses of diets and feeding trial responses may be completed with an a priori .alpha.=0.05. Analysis of performance parameters among treatments may be performed with appropriate analysis of variance or covariance (Proc Mixed) and post hoc multiple comparisons, as needed. Analysis of fish performance and tissue responses may be assessed by nonlinear models.

In embodiments, the present disclosure proposes to convert fibers and other carbohydrates in canola into additional protein using, for example, a GRAS-status microbe. A microbial exopolysaccharide (i.e., gum) may also be produced that may facilitate extruded feed pellet formation, negating the need for binders. This microbial gum may also provide immunostimulant activity to activate innate defense mechanisms that protect fish from common pathogens resulting from stressors. Immunoprophylactic substances, such as β-glucans, bacterial products, and plant constituents, are increasingly used in commercial feeds to reduce economic losses due to infectious diseases and minimize antibiotic use. The microbes of the present disclosure also produce extracellular peptidases, which should increase corn protein digestibility and absorption during metabolism, providing higher feed efficiency and yields. As disclosed herein, this microbial incubation process provides a valuable, sustainable aquaculture feed that is less expensive per unit of protein than CBPP and fish meal.

After extrusion pretreatment, cellulose-deconstructing enzymes may be evaluated to generate sugars, which microbes of the present disclosure may convert to protein and gum. In embodiments, sequential omission of these enzymes and evaluation of co-culturing with cellulolytic microbes may be used. Ethanol may be evaluated to precipitate the gum and improve centrifugal recovery of the CBPP. After drying, the CBPP may be incorporated into practical diet formulations. In embodiments, test grower diets may be formulated (with mineral and vitamin premixes) and comparisons to a fish-meal control and commercial canola based protein product (CBPP), as it contains traces of oligopolysaccharides, GLS, and antigenic substances diets in feeding trials with a commercially important fish, e.g., yellow perch or rainbow trout, may be performed. Performance (e.g., growth, feed conversion, protein efficiency), viscera characteristics, and intestinal histology may be examined to assess fish responses.

In other embodiments, optimizing the canola meal-based protein production process by determining optimum pretreatment and conversion conditions while minimizing process inputs, improving the performance and robustness of the microbe, testing the resultant grower feeds for a range of commercially important fishes, validating process costs and energy requirements, and completing initial steps for scale-up and commercialization may be carried out.

Fish that can be fed the fish feed composition of the present disclosure include, but are not limited to, Siberian sturgeon, Sterlet sturgeon, Starry sturgeon, White sturgeon, Arapaima, Japanese eel, American eel, Short-finned eel, Long-finned eel, European eel, Chanos chanos, Milkfish, Bluegill sunfish, Green sunfish, White crappie, Black crappie, Asp, Catla, Goldfish, Crucian carp, Mud carp, Mrigal carp, Grass carp, Common carp, Silver carp, Bighead carp, Orangefin labeo, Roho labeo, Hoven's carp, Wuchang bream, Black carp, Golden shiner, Nilem carp, White amur bream, That silver barb, Java, Roach, Tench, Pond loach, Bocachico, Dorada, Cachama, Cachama Blanca, Paco, Black bullhead, Channel catfish, Bagrid catfish, Blue catfish, Wels catfish, Pangasius (Swai, Tra, Basa) catfish, Striped catfish, Mudfish, Philippine catfish, Hong Kong catfish, North African catfish, Bighead catfish, Sampa, South American catfish, Atipa, Northern pike, Ayu sweetfish, Vendace, Whitefish, Pink salmon, Chum salmon, Coho salmon, Masu salmon, Rainbow trout, Sockeye salmon, Chinook salmon, Atlantic salmon, Sea trout, Arctic char, Brook trout, Lake trout, Atlantic cod, Pejerrey, Lai, Common snook, Barramundi/Asian sea bass, Nile perch, Murray cod, Golden perch, Striped bass, White bass, European seabass, Hong Kong grouper, Areolate grouper, Greasy grouper, Spotted coralgrouper, Silver perch, White perch, Jade perch, Largemouth bass, Smallmouth bass, European perch, Zander (Pike-perch), Yellow Perch, Sauger, Walleye, Bluefish, Greater amberjack, Japanese amberjack, Snubnose pompano, Florida pompano, Palometa pompano, Japanese jack mackerel, Cobia, Mangrove red snapper, Yellowtail snapper, Dark seabream, White seabream, Crimson seabream, Red seabream, Red porgy, Goldlined seabream, Gilthead seabream, Red drum, Green terror, Blackbelt cichlid, Jaguar guapote, Mexican mojarra, Pearlspot, Three spotted tilapia, Blue tilapia, Longfin tilapia, Mozambique tilapia, Nile tilapia, Tilapia, Wami tilapia, Blackchin tilapia, Redbreast tilapia, Redbelly tilapia, Golden grey mullet, Largescale mullet, Gold-spot mullet, Thinlip grey mullet, Leaping mullet, Tade mullet, Flathead grey mullet, White mullet, Lebranche mullet, Pacific fat sleeper, Marble goby, White-spotted spinefoot, Goldlined spinefoot, Marbled spinefoot, Southern bluefin tuna, Northern bluefin tuna, Climbing perch, Snakeskin gourami, Kissing gourami, Giant gourami, Snakehead, Indonesian snakehead, Spotted snakehead, Striped snakehead, Turbot, Bastard halibut (Japanese flounder), Summer Flounder, Southern flounder, Winter flounder, Atlantic Halibut, Greenback flounder, Common sole, crustaceans and combinations thereof.

It will be appreciated by the skilled person that the fish feed composition of the present disclosure may be used as a convenient carrier for pharmaceutically active substances such as for example antimicrobial agents and immunologically active substances including vaccines against bacterial or viral infections, and any combination thereof.

The fish feed composition according to present disclosure may be provided as a liquid, pourable emulsion, or in the form of a paste, or in a dry form, for example as a granulate or pellet, a powder, or as flakes. When the fish feed composition is provided as an emulsion, a lipid-in-water emulsion, it is may be in a relatively concentrated form. Such a concentrated emulsion form may also be referred to as a pre-emulsion as it may be diluted in one or more steps in an aqueous medium to provide the final enrichment medium for the organisms.

Another aspect of the present invention is directed towards complete fish meal compositions with an enhanced concentration of nutrients which includes microorganisms characterized by an enhanced concentration of nutrients such as, but not limited to, fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin, silicon, and combinations thereof.

In an incubation process of the present disclosure, a carbon source may be hydrolyzed to its component sugars by microorganisms to produce alcohol and other gaseous products. Gaseous product includes carbon dioxide and alcohol includes ethanol. The incubation products obtained after the incubation process are typically of higher commercial value. In embodiments, the incubation products contain microorganisms that have enhanced nutrient content than those products deficient in the microorganisms. The microorganisms may be present in an incubation system, the incubation broth and/or incubation biomass. The incubation broth and/or biomass may be dried (e.g., spray-dried), to produce the incubation products with an enhanced content of the nutritional contents.

The nutrient enriched incubation product of this disclosure may have a nutrient content of from at least about 1% to about 95% by weight. The nutrient content is preferably in the range of at least about 10%-20%, 20%-30%, 30%-40%, 40%-50%, 50%-60%, 60%-70%, and 70%-80% by weight. The available nutrient content may depend upon the animal to which it is fed and the context of the remainder of the diet, and stage in the animal life cycle. For instance, beef cattle require less histidine than lactating cows. Selection of suitable nutrient content for feeding animals is well known to those skilled in the art.

The incubation products may be prepared as a spray-dried biomass product. Optionally, the biomass may be separated by known methods, such as centrifugation, filtration, separation, decanting, a combination of separation and decanting, ultrafiltration or microfiltration. The biomass incubation products may be further treated to facilitate rumen bypass. In embodiments, the biomass product may be separated from the incubation medium, spray-dried, and optionally treated to modulate rumen bypass, and added to feed as a nutritional source. In addition to producing nutritionally enriched incubation products in a incubation process containing microorganisms, the nutritionally enriched incubation products may also be produced in transgenic plant systems. Methods for producing transgenic plant systems are known in the art. Alternatively, where the microorganism host excretes the nutritional contents, the nutritionally-enriched broth may be separated from the biomass produced by the incubation and the clarified broth may be used as an animal feed ingredient, e.g., either in liquid form or in spray dried form.

The incubation products obtained after the incubation process using microorganisms may be used as an animal feed or as food supplement for humans. The incubation product includes at least one ingredient that has an enhanced nutritional content that is derived from a non-animal source (e.g., a bacteria, yeast, and/or plant). In particular, the incubation products are rich in at least one or more of fats, fatty acids, lipids such as phospholipid, vitamins, essential amino acids, peptides, proteins, carbohydrates, sterols, enzymes, and trace minerals such as, iron, copper, zinc, manganese, cobalt, iodine, selenium, molybdenum, nickel, fluorine, vanadium, tin and silicon. In embodiments, the peptides contain at least one essential amino acid. In other embodiments, the essential amino acids are encapsulated inside a subject modified microorganism used in an incubation reaction. In embodiments, the essential amino acids are contained in heterologous polypeptides expressed by the microorganism. Where desired, the heterologous polypeptides are expressed and stored in the inclusion bodies in a suitable microorganism (e.g., fungi).

In embodiments, the incubation products have a high nutritional content. As a result, a higher percentage of the incubation products may be used in a complete animal feed. In embodiments, the feed composition comprises at least about 15% of incubation product by weight. In a complete feed, or diet, this material will be fed with other materials. Depending upon the nutritional content of the other materials, and/or the nutritional requirements of the animal to which the feed is provided, the modified incubation products may range from 15% of the feed to 100% of the feed. In embodiments, the subject incubation products may provide lower percentage blending due to high nutrient content. In other embodiments, the subject incubation products may provide very high fraction feeding, e.g. over 75%. In suitable embodiments, the feed composition comprises at least about 20%, at least about 25%, at least about 30%, at least about 35%, at least about 40%, at least about 45%, at least about 50%, at least about 60%, at least about 70%, or at least about 75% of the subject incubation products. Commonly, the feed composition comprises at least about 20% of incubation product by weight. More commonly, the feed composition comprises at least about 15-25%, 25-20%, 20-25%, 30%-40%, 40%-50%, 50%-60%, or 60%-70% by weight of incubation product. Where desired, the subject incubation products may be used as a sole source of feed.

The complete fish meal compositions may have enhanced amino acid content with regard to one or more essential amino acids for a variety of purposes, e.g., for weight increase and overall improvement of the animal's health. The complete fish meal compositions may have an enhanced amino acid content because of the presence of free amino acids and/or the presence of proteins or peptides including an essential amino acid, in the incubation products. Essential amino acids may include arginine, cysteine, histidine, isoleucine, lysine, methionine, phenylalanine, threonine, taurine, tryptophan, and/or valine, which may be present in the complete animal feed as a free amino acid or as part of a protein or peptide that is rich in the selected amino acid. At least one essential amino acid-rich peptide or protein may have at least 1% essential amino acid residues per total amino acid residues in the peptide or protein, at least 5% essential amino acid residues per total amino acid residues in the peptide or protein, or at least 10% essential amino acid residues per total amino acid residues in the protein. By feeding a diet balanced in nutrients to animals, maximum use is made of the nutritional content, requiring less feed to achieve comparable rates of growth, milk production, or a reduction in the nutrients present in the excreta reducing bioburden of the wastes.

A complete fish meal composition with an enhanced content of an essential amino acid, may have an essential amino acid content (including free essential amino acid and essential amino acid present in a protein or peptide) of at least 2.0 wt % relative to the weight of the crude protein and total amino acid content, and more suitably at least 5.0 wt % relative to the weight of the crude protein and total amino acid content. The complete fish meal composition includes other nutrients derived from microorganisms including but not limited to, fats, fatty acids, lipids such as phospholipid, vitamins, carbohydrates, sterols, enzymes, and trace minerals.

The complete fish meal composition may include complete feed form composition, concentrate form composition, blender form composition, and base form composition. If the composition is in the form of a complete feed, the percent nutrient level, where the nutrients are obtained from the microorganism in an incubation product, which may be about 10 to about 25 percent, more suitably about 14 to about 24 percent; whereas, if the composition is in the form of a concentrate, the nutrient level may be about 30 to about 50 percent, more suitably about 32 to about 48 percent. If the composition is in the form of a blender, the nutrient level in the composition may be about 20 to about 30 percent, more suitably about 24 to about 26 percent; and if the composition is in the form of a base mix, the nutrient level in the composition may be about 55 to about 65 percent. Unless otherwise stated herein, percentages are stated on a weight percent basis. If the HQPC is high in a single nutrient, e.g., Lys, it will be used as a supplement at a low rate; if it is balanced in amino acids and Vitamins, e.g., vitamin A and E, it will be a more complete feed and will be fed at a higher rate and supplemented with a low protein, low nutrient feed stock, like corn stover.

The fish meal composition may include a peptide or a crude protein fraction present in an incubation product having an essential amino acid content of at least about 2%. In embodiments, a peptide or crude protein fraction may have an essential amino acid content of at least about 3%, at least about 5%, at least about 10%, at least about 15%, at least about 20%, at least about 30%, at least about 40%, and in embodiments, at least about 50%. In embodiments, the peptide may be 100% essential amino acids. Commonly, the fish meal composition may include a peptide or crude protein fraction present in an incubation product having an essential amino acid content of up to about 10%. More commonly, the fish meal composition may include a peptide or a crude protein fraction present in an incubation product having an essential amino acid content of about 2-10%, 3.0-8.0%, or 4.0-6.0%.

The fish meal composition may include a peptide or a crude protein fraction present in a incubation product having a lysine content of at least about 2%. In embodiments, the peptide or crude protein fraction may have a lysine content of at least about 3%, at least about 5%, at least about 10%, at least about 15%, at least about 20%, at least about 30%, at least about 40%, and in embodiments, at least about 50%. Typically, the fish meal composition may include the peptide or crude protein fraction having a lysine content of up to about 10%. Where desired, the fish meal composition may include the peptide or a crude protein fraction having a lysine content of about 2-10%, 3.0-8.0%, or 4.0-6.0%.

The fish meal composition may include nutrients in the incubation product from about 1 g/Kg dry solids to 900 g/Kg dry solids. In embodiments, the nutrients in a fish meal composition may be present to at least about 2 g/Kg dry solids, 5 g/Kg dry solids, 10 g/Kg dry solids, 50 g/Kg dry solids, 100 g/Kg dry solids, 200 g/Kg dry solids, and about 300 g/Kg dry solids. In embodiments, the nutrients may be present to at least about 400 g/Kg dry solids, at least about 500 g/Kg dry solids, at least about 600 g/Kg dry solids, at least about 700 g/Kg dry solids, at least about 800 g/Kg dry solids and/or at least about 900 g/Kg dry solids.

The fish meal composition may include an essential amino acid or a peptide containing at least one essential amino acid present in an incubation product having a content of about 1 g/Kg dry solids to 900 g/Kg dry solids. In embodiments, the essential amino acid or a peptide containing at least one essential amino acid in a fish meal composition may be present to at least about 2 g/Kg dry solids, 5 g/Kg dry solids, 10 g/Kg dry solids, 50 g/Kg dry solids, 100 g/Kg dry solids, 200 g/Kg dry solids, and about 300 g/Kg dry solids. In embodiments, the essential amino acid or a peptide containing at least one essential amino acid may be present to at least about 400 g/Kg dry solids, at least about 500 g/Kg dry solids, at least about 600 g/Kg dry solids, at least about 700 g/Kg dry solids, at least about 800 g/Kg dry solids and/or at least about 900 g/Kg dry solids.

The complete fish meal composition may contain a nutrient enriched incubation product in the form of a biomass formed during incubation and at least one additional nutrient component. In another example, the fish meal composition contains a nutrient enriched incubation product that is dissolved and suspended from an incubation broth formed during incubation and at least one additional nutrient component. In a further embodiment, the fish meal composition has a crude protein fraction that includes at least one essential amino acid-rich protein. The fish meal composition may be formulated to deliver an improved balance of essential amino acids.

Highly unsaturated fatty acids (HUFAs) in microorganisms, when exposed to oxidizing conditions may be converted to less desirable unsaturated fatty acids or to saturated fatty acids. However, saturation of omega-3 HUFAs may be reduced or prevented by the introduction of synthetic antioxidants or naturally-occurring antioxidants, such as beta-carotene, vitamin E and vitamin C, into the feed. Synthetic antioxidants, such as BHT, BHA, TBHQ or ethoxyquin, or natural antioxidants such as tocopherols, may be incorporated into the food or feed products by adding them to the products, or they may be incorporated by in situ production in a suitable organism. The amount of antioxidants incorporated in this manner depends, for example, on subsequent use requirements, such as product formulation, packaging methods, and desired shelf life.

Incubation products or complete fish meal containing the incubation products of the present disclosure, may also be utilized as a nutritional supplement for human consumption if the process begins with human grade input materials, and human food quality standards are observed through out the process. Incubation product or the complete feed as disclosed herein is high in nutritional content. Nutrients such as, protein and fiber are associated with healthy diets. Recipes may be developed to utilize incubation product or the complete feed of the disclosure in foods such as cereal, crackers, pies, cookies, cakes, pizza crust, summer sausage, meat balls, shakes, and in any forms of edible food. Another choice may be to develop the incubation product or the complete feed of the disclosure into snacks or a snack bar, similar to a granola bar that could be easily eaten, convenient to distribute. A snack bar may include protein, fiber, germ, vitamins, minerals, from the grain, as well as nutraceuticals such as glucosamine, HUFAs, or co-factors, such as Vitamin Q-10.

The fish meal comprising the subject incubation products may be further supplemented with flavors. The choice of a particular flavor will depend on the animal to which the feed is provided. The flavors and aromas, both natural and artificial, may be used in making feeds more acceptable and palatable. These supplementations may blend well with all ingredients and may be available as a liquid or dry product form. Suitable flavors, attractants, and aromas to be supplemented in the animal feeds include but not limited to fish pheromones, fenugreek, banana, cherry, rosemary, cumin, carrot, peppermint oregano, vanilla, anise, plus rum, maple, caramel, citrus oils, ethyl butyrate, menthol, apple, cinnamon, any natural or artificial combinations thereof. The favors and aromas may be interchanged between different animals. Similarly, a variety of fruit flavors, artificial or natural may be added to food supplements comprising the subject incubation products for human consumption.

The shelf-life of the incubation product or the complete feed of the present disclosure may typically be longer than the shelf life of an incubation product that is deficient in the microorganism. The shelf-life may depend on factors such as, the moisture content of the product, how much air can flow through the feed mass, the environmental conditions and the use of preservatives. A preservative may be added to the complete feed to increase the shelf life to weeks and months. Other methods to increase shelf life include management similar to silage management such as mixing with other feeds and packing, covering with plastic or bagging. Cool conditions, preservatives and excluding air from the feed mass all extend shelf life of wet co-products. The complete feed can be stored in bunkers or silo bags. Drying the wet incubation product or complete feed may also increase the product's shelf life and improve consistency and quality.

The complete feed of the present disclosure may be stored for long periods of time. The shelf life may be extended by ensiling, adding preservatives such as organic acids, or blending with other feeds such as soy hulls. Commodity bins or bulk storage sheds may be used for storing the complete feeds.

In embodiments a solid state incubation method is disclosed that does not require mixing the moist solid substrate. In one aspect, instead of mixing the solid substrate (which takes a high amount of energy), this method as disclosed pelletizes the substrate so that it can be placed in static beds, and then passing moist air through the void spaces between the pellets to control temperature, aeration, and moisture levels.

Most microbial conversion processes are conducted in a submerged state, with the substrate mixed with water to achieve up to 25-30% solids (70-75% moisture). This flowable, mixable slurry can be readily processed in traditional stirred tank reactors in which mass transfer and control of process conditions can be readily achieved. The key disadvantage of submerged conversion is the high amount of water needed. This dilutes the product, resulting in large reactor volumes, and a large amount of water that must ultimately be removed and processed.

Solid state incubation is conducted at 30-70% moisture, where the substrate is in the form of a moist solid. Microbes grow on the film of water on the surface of the particles. This process requires less water and reactor sizes can be smaller. However the chief challenge with solid state incubation is in achieving acceptable mass transfer and control of operating conditions (temperature, aeration, moisture, pH, etc.).

Mixed solid state reactors have used paddles or auger type systems that mix the entire contents continuously. This takes a high amount of energy, and may be disadvantageous for many types of fungi that prefer to grow in filaments under static condition. For example, the Nauta type mixer uses an smaller auger that gradually moves around the inside of a cone shaped reactor. Only a portion of the solids are moved at any one time, thus greatly reducing energy usage.

Typically, static solid state incubation has used shallow beds of the feedstock (inches in depth), so that air can pass through the bed and heat can be easily dissipated. The degree of air movement depends on the moisture content and particle size of the substrate, along with the degree to which the microbial growth fills in the void spaces. The disadvantage here is that it is difficult to automate “bed or tray” type systems, and there is a relatively large amount of un-utilized space above each bed or tray.

The method as disclosed herein is an adaptation of the “bed or tray” type system, except that instead of using loose substrate, the substrate is first formed into pellets or “callets” that are formed by pressing or extruding the substrate. The larger the pellets, the greater the void space in the bed, and the greater the potential air movement.

A secondary innovation is the use of a novel “atomization” process which creates small droplets of inoculum for maximum coverage. This greatly reduces the amount of inoculum needed.

Submerged incubation processes are used for the vast majority of aerobic and anaerobic microbial conversions. Silage fermentations in bunkers or long plastic bags are good examples of a static, anaerobic microbial process. Mushroom production is an example of a static, aerobic microbial process. Composting is an example of a periodically-mixed, aerobic microbial process. All of these typically use a mixed community of microbes, with the goal of degrading lignocellulosic type feedstocks.

In embodiments, roughly marble sized pellets of feedstock are formed which contain 30-50% moisture and a water activity of less than about 0.7.

The pellets may be formed through an extrusion processes, and may use sterilized or pasteurized pellets. In one aspect, binding agent may be added to the feedstock to aid in pellet formation. In one aspect, the pellets are strong enough to support the weight of additional pellets stacked on top of them, and yet porous enough to allow easier microbial colonization and air flow into the pellets.

In one aspect, the pellets may be transported under a UV light by conveyor to a static bioreactor containing an auger mechanism in the bottom to remove the feedstock after incubation. The reactor may be operated in batch or continuous mode.

After cooling, the pellets may be inoculated by a fogging system that would allow “atomization” of a very dilute liquid inoculum that may be spread throughout the entire pellet bed, and also into the pellets having adequate porosity. The incubator may be pulsed with fresh inoculum at set intervals to, not only inoculate fresh material, but to maintain humidity and support existing colonization throughout the reactor. The fogging system may consist of an atomizer with the capabilities of using high frequency waves to create a suspension of inoculum inside small droplets. The fogging system may also have the ability to maintain a stable temperature and rocking motion to maximize time between adding new inoculum material.

In embodiments, during incubation the fungus will colonize the surface of the pellets and grow throughout the interior of the pellets. Using the atomized fog to control humidity and ensure even colonization, material near the bottom of the reactor will have completed the incubation process, while new material near the top will be freshly inoculated. In one aspect, the method comprises a continuous flow reactor in which new pellets are added on the top, and get “buried” progressively deeper each day as old material is withdrawn from the bottom and new material is added to the top. As the pellets get colonized, their physical strength increases, which will ensure integrity as they complete fermentation under the weight of new pellets.

The risk of the pellets knitting together is that bridging may occur in the reactor, preventing the material from slowly falling downward. In embodiments, the reactor is constructed so that it has larger dimensions at the bottom compared to the top. The bottom auger (or augers) may be designed so they can efficiently break apart and withdraw the final material.

After incubation, the pellets may be augered out of the reactor, put into a dryer, and milled when adequately dried.

Other potential applications of this technology could be in feed manufacturing processes that use aerobic microbes. Most feed processes that currently use solid stat incubation are anaerobic (e.g., Silage fermentation) and these primarily convert sugars in the feedstock into organic acids to act as a preservative. These systems do not significantly improve the susceptibility of the lignocellulose feedstock to digestion. Using the process as disclosed herein, the lignocellulose feedstock could be inoculated with cellulase producing fungi that would enhance subsequent digestibility in the livestock.

The atomizer application may be utilized as method for inoculation in any process requiring microbial conversion. The entire system may be modified to fit different microbial conversions.

As used herein, “room temperature” is about 25° C. under standard pressure.

The following examples are intended to illustrate but not limit the invention.

### EXAMPLES

### Methods

### Feedstocks and Preparation

Hexane extracted (HE) canola meal was obtained from North Dakota State University (Fargo, N. Dak., USA), while cold pressed (CP) canola meal was obtained from Agrisoma Biosciences (Ottawa, Ontario, Canada). Both HE and CP meals were milled through a 2 mm screen via knife mill prior to use, and were stored at room temperature in sealed buckets throughout the duration of experimentation. Dry weight (dw) analysis was conducted by drying ˜5 grams of canola meal at 80° C. in a drying oven for at least 48 h. Proximate analysis was conducted following A.O.A.C. protocols by SGS (Brookings, S. Dak., USA), and Table 1 provides the composition of each feedstock.

TABLE 1
Proximate analysis of hexane extracted and cold pressed canola
meal before and after processing with A. pullulans (Y-2311-1)

| | | |
|---|---|---|
| | Hexane | |
| Analysis | Extracted | Cold Pressed |

| (% w/w, db) | Raw | Processed | Raw | Processed |
|---|---|---|---|---|
| Dry Matter | 93.5 | 96.1 | 93.7 | 96.9 |
| Crude Ash | 7.5 | 8.7 | 6.8 | 7.2 |
| Crude Fat | 5.4 | 5.7 | 17.1 | 18.6 |
| Crude Fiber | 12.4 | 13.5 | 7.3 | 7.9 |
| Crude Protein | 40.4 | 44.1 | 43.7 | 48.1 |

### Cultures, Maintenance, and Inoculum Preparation

Aureobasidium pullulans (NRRL-58522), A. pullulans (NRRL-42023), A. pullulans (NRRL-Y-2311-1), Trichoderma reesei (NRRL-3653) and Fusarium venenatum (NRRL-26139) were obtained from the National Center for Agricultural Utilization Research (Peoria, Ill.). Pichia kudriavzevii and Mucor circinelloides were isolated as contaminates from prior trials, and were identified by ARS-USDA (Peoria, Ill., USA) using 15s RNA analysis. Short-term maintenance cultures were stored on Potato Dextrose Agar (PDA) plates and slants at 4 degrees Celsius (° C.). Lyophilization was used for long-term storage. Inocula for all experiments were prepared by transferring isolated colonies or a square section of agar growth (filamentous fungi) into glucose yeast extract (GYE) medium consisting of 5% glucose and 0.5% yeast extract. The pH for Aureobasidium, Pichia, and Mucor cultures was adjusted to 3 with 10N sulfuric acid, while pH 5-5.5 was used for Trichoderma and Fusarium. GYE flasks consisted of 100 milliliter (ml) working volume in 250 ml Erlenmeyer flasks, covered with a foam plug and aluminum foil. Cultures were incubated for ˜72 hours (h) at 30° C. in a rotary shaker at 150 revolutions per minute (rpm).

### Pretreatments

Extrusion included a barrel temperature of 80° C., and a screw speed of 50 or 100 rpm for CP and HE canola meals, respectively. Extrusion was completed at meal moisture contents of 4.6 and 7.3% for CP and HE canola meals, respectively. Extruded material was stored in a sealed bucket until trials were completed. Hot water cook, dilute acid, and dilute alkali pretreatments were conducted on homogenized, 15% w/w (dmb) slurries of CP and HE canola meals at 160° C. for 20 min using a stainless steel steam jacketed reactor tune in 8-10 1 batch-wise increments. Dilute acid pretreatment also incorporated 0.5% w/w sulfuric acid, while dilute alkali pretreatment used a 4% w/w ammonia concentration (using 30% ammonium hydroxide). Following pretreatment, these slurries were frozen for storage. Prior to use, slurries were thawed, re-homogenized, and evenly dispersed into flasks for various trials.

### Solid State Trials

Solid state trials were conducted in 500 ml Erlenmeyer flasks with 100 g of 50% moisture content canola meal (hexane extracted vs. cold pressed). Flask contents were pH adjusted with 10 N sulfuric acid to the optimum pH for each organism. Flasks were covered with foam plugs and aluminum foil and were then autoclaved at 121° C. for 20 min. Flasks were inoculated with 10 ml of ˜72 h inoculum cultures, then incubated statically at 30° C. for 168 h. Visual subjective rating of colonization percentages on surfaces were conducted daily. Following incubation the solids were recovered, the pH was measured, and the solids were then dried and analyzed for carbohydrates, protein, fiber, and glucosinolates.

### Submerged Trials

Submerged trials were conducted in 1 L Erlenmeyer flasks with 500 ml total volume at 10% solid loading rate (SLR) dry weight canola meal. Flasks were covered with foam plugs and aluminum foil. For trials to be subjected to initial saccharification step, 10N sulfuric acid was used to adjust the initial pH to 5 (this is the optimal level for the commercial cellulase and hemicellulase enzymes used). For trials lacking the saccharification step, the pH was adjusted to the levels indicated previously for specific microbes. Flasks were then autoclaved at 121 C.° for 20 min. For saccharification trials, 0.052 ml CTEC2 and 0.138 ml HTEC2 (Novozymes, Franklin, N.C.) were added, and flasks were incubated at 50° C. and 150 RPM for 24 h. Following saccharification, the pH was adjusted (if necessary) for the specific microbes and the slurry was cooled to 30° C. Saccharification and non-saccharification trials were inoculated with 5 ml of a 72 h culture of the appropriate organism and incubated at 30° C. at 150 RPM for 168 h. Daily samples of ˜50 ml were collected and used to monitor pH, cell counts, carbohydrates, protein fiber, and GLS as described herein. At the end of incubation, the slurry was dried for 2 d at 80° C.

### Analytical Methods

After 168 h incubation, the pH of each sample was measured (Oakton pH Spear). The solids were then dried for 2 d at 80° C. One g of each dried sample was removed and mixed with 9 ml diH₂O, then allowed to solubilize at 4° C. overnight. This solution was then centrifuged at 10,000 RPM for 10 min and the supernatant was then poured into a 2 ml microcentrifuge tube and frozen overnight. After thawing, the supernatant was centrifuged a second time at 10,000 RPM for 10 min to remove any precipitants, and this supernatant was then filtered through a 0.2 μm filter and into a HPLC vial. A Waters size-exclusion chromatography column (SugarPak column I with pre-column module, Waters Corporation, Milford, Mass., USA) and high performance liquid chromatography system (Agilent Technologies, Santa Clara, Calif., USA) equipped with refractive index detector (Model G1362A) were used to measure the sugars. The sugars were eluted using a de-ionized water as mobile phase at flow rate of 0.5 mL/min and column temperature of 80° C. Sugars quantified included arabinose, galactose, glucose, raffinose, stachyose, and sucrose.

### Total Protein

Approximately 5 g of sample was used for protein and glucosinolate (GLS) analysis. Protein was quantified using a LECO model FP528 (St. Joseph, Mich., USA) to combust the sample, measure the nitrogen gas content, and then calculate protein percentage. Protein percentage was calculated from the nitrogen content of the sample using a conversion factor of 6.25.

### Glucosinolates

Total and individual GLS products were quantified using reverse phase high performance liquid chromatography (RP-HPLC) and identified using quadrupole time-of-flight (Q-TOF) liquid chromatography-mass spectrometry (LC-MS). For GLS quantification, a modification of an HPLC method developed by Betz and Fox (High-Performance liquid chromatographic determination of glucosinolates in Brassica vegetables. In Huang et al. (eds.), Food Phytochemicals I: Fruits and Vegetables. ACS Symposium Series, American Chemical Society, Washington D.C., USA, pp. 181-196 (1994)) was used. The extract was run on a Shimadzu (Columbia, Md.). HPLC system (two LC 20AD pumps; SIL 20A autoinjector; DGU 20 As degasser; SPD-20A UV-Vis detector; and a CBM-20A communication BUS module) running under the Shimadzu LC solutions Version 1.25 software. The column was a C18 Inertsil reverse phase column (250×4.6 mm; RP C-18, ODS-3, 5μ; with a Metaguard guard column; Varian, Torrance, Calif.). The GLS were detected by monitoring at 237 nm. Initial mobile phase conditions were 12% methanol/88% aqueous 0.005-M tetrabutylammonium bisulfate (TBS) at a flow rate of 1 ml/min. After injection of 15 μl of sample, the initial conditions were held for 2 min, and then up to 35% methanol over another 20 min, the up to 100% methanol over another 10 min.

### Fiber

Fiber analysis was completed as Neutral Detergent Fiber (NDF) and Acid Detergent fiber (ADF). NDF is a method commonly used for animal feed analysis to determine the amount of lignin, hemicellulose and cellulose while ADF represents the least digestible fiber fraction of animal feed including lignin, cellulose, silica but not hemicellulose. NDF and ADF analysis were completed by Midwest Laboratories (Omaha, Nebr., USA) using ANKOM Technology (Macedon, N.Y., USA) filter bag methods.

### Example 1

### Solid State Trials

Seven fungal strains were grown on hexane extracted (HE) vs. cold pressed (CP) canola meal using a solid state incubation process. As listed in Table 1, the composition of HE and CP meals were different in terms of the fat and fiber content. It was expected that GLS in both feedstocks (42.8-60.6 μM/g), or the higher oil content of CP canola meal, to be inhibitory. It is art recognized that GLS may inhibit some types of microbes. It was also expected that canola oil would be inhibitory, as it is art recognized that high oil concentrations can reduce microbial growth. Cold pressing typically removes only 75-85% of canola seed oil, while solvent extraction removes greater than 96%.

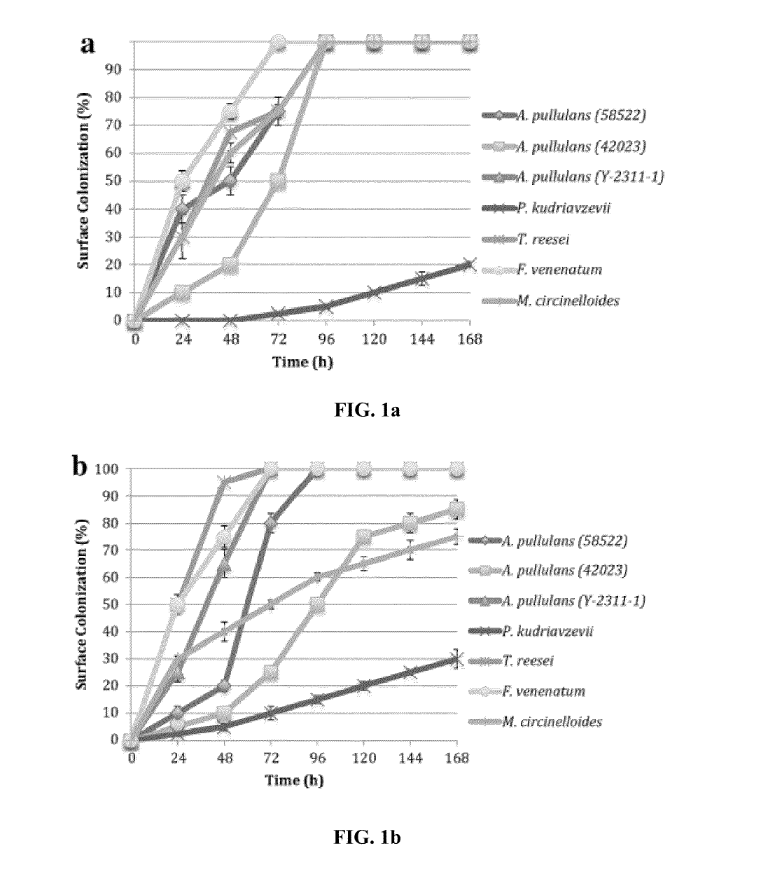

When the microbial strains were cultivated in those meals, a different trend of colonization was observed. FIGS. 1a and b show the percent surface colonization for each strain during incubation of HE and CP canola meal, respectively. While these visual ratings were subjective, they do provide an indication of relative growth rates on the two feedstocks.

FIG. 1a shows that A. pullulans (NRRL-Y-2311-1) and F. venenatum grew the most rapidly on HE canola meal, achieving 100% colonization in 72 h. P. kudriavzevii grew the slowest, only achieving 20% colonization in 168 h. FIG. 1b shows that A. pullulans (NRRL-Y-2311-1), F. venenatum, and T. reesei grew the most rapidly on CP canola meal, achieving 100% colonization in 72 h. P. kudriavzevii again grew the slowest, only achieving 30% colonization in 168 h. Therefore the higher oil content of cold pressed canola meal did not affect growth of A. pullulans (NRRL-Y-2311-1) and F. venenatum, and actually stimulated growth of T. reesei. The extra oil also improved the growth of A. pullulans (NRRL-42023) and M. circinelloides, perhaps by providing an additional carbon and energy source. The slower colonization rate and extent of P. kudriavzevii on both feedstocks was expected, as it is a single-celled yeast that does not grow in a filamentous morphology. It was included in these trials because it frequently occurs as a contaminant in larger scale trials where processing plant-based protein meals is carried out in submerged conditions. In broth culture P. kudriavzevii exhibits a much faster growth rate of >0.30 h⁻¹ compared to the filamentous fungi.

Table 2 shows the initial versus final pH levels for these trials.

TABLE 2
Initial versus final pH of hexane extracted and
cold pressed canola meal

| | Optimal | Initial pH | Final pH | | |
|---|---|---|---|---|---|
| | pH | HE | HE | Initial pH | Final pH |
| Fungal Culture | Range | Canola | Canola | CP Canola | CP Canola |
| Control | — | 3.2 ± 0.0 | 3.2 ± 0.0 | 2.9 ± 0.1 | 3.0 ± 0.1 |
| A. pullulans | 3.0-5.0 | 3.0 ± 0.0 | 3.8 ± 0.5 | 3.1 ± 0.1 | 3.9 ± 0.4 |
| (NRRL-58522) | | | | | |
| A. pullulans | 3.0-5.0 | 3.1 ± 0.0 | 3.7 ± 0.1 | 3.0 ± 0.0 | 3.0 ± 0.4 |
| (NRRL-42023) | | | | | |
| A. pullulans | 3.0-5.0 | 3.2 ± 0.2 | 6.2 ± 0.4 | 3.1 ± 0.1 | 4.9 ± 0.6 |
| (NRRL-Y- | | | | | |
| 2311-1) | | | | | |
| P. kudriavzevii | 3.0 | 3.0 ± 0.0 | 3.7 ± 0.3 | 3.0 ± 0.1 | 3.1 ± 0.2 |
| T. reesei | 4.0-6.0 | 5.0 ± 0.0 | 7.7 ± 0.1 | 5.0 ± 0.1 | 7.1 ± 0.3 |
| (NRRL-3653) | | | | | |
| F. venenatum | 4.5-6.0 | 5.1 ± 0.3 | 7.4 ± 0.4 | 5.1 ± 0.1 | 6.2 ± 0.7 |
| (NRRL-26139) | | | | | |
| M. | 3.0-6.0 | 3.1 ± 0.1 | 4.9 ± 0.7 | 3.1 ± 0.1 | 3.9 ± 0.8 |
| circinelloides | | | | | |

The pH in the un-inoculated controls stayed stable throughout 168 h incubation, and the pH in trials with A. pullulans (NRRL-58522), A. pullulans (NRRL-42023), and P. kudriavzevii increased by less than 1 pH unit. The pH increased from 3.0 to 3.9-4.9 in the M. circinelloides trials, however studies have shown this fungus has a broad pH range of 3.0-8.0. In trials with A. pullulans (NRRL-Y-2311-1), T. reesei, and F. venenatum, the pH also rose, in some cases to slightly above the optimal range. Nevertheless, these strains still exhibited the most rapid colonization rates in both HE and CP canola meals.

In most cases the pH rose to a higher level in HE canola meal compared to CP meal. Due to the fact that these solid-state trials were not mixed or sampled until the end of incubation, it was not possible to adjust pH during incubation. However, it is possible that the increased pH might have affected fungal metabolism, and hence protein production. pH may be controlled in a paddle-type reactor (data not shown).

### Total Proteins

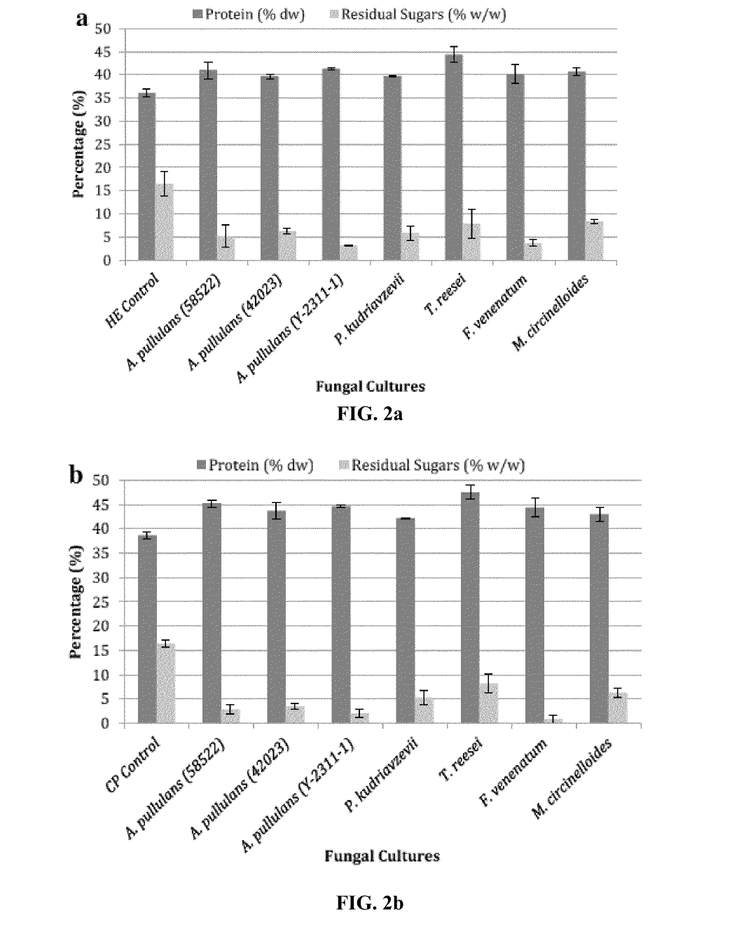

FIGS. 2a and 2b present the maximum protein levels and residual sugar levels in HE and CP canola meals, respectively, for the un-inoculated control versus the various fungi. Protein levels increased from 36.1% in hexane extracted meal to 39.7-44.4% after solid-state microbial conversion, representing relative improvements of ˜10-23%. Protein levels increased from 38.6% in cold pressed meal to 42.2-47.5% after solid-state microbial conversion (relative improvements of ˜9-23%). T. reesei achieved the highest protein levels for both substrates, while P. kudriavzevii exhibited the lowest protein enhancement of all strains. T. reesei is known to produce many hydrolytic enzymes, and was expected to provide the greatest conversion of fiber and oligosaccharides into cell mass. As a single-celled yeast, P. kudriavzevii does not produce cellulase enzymes and was therefore anticipated to result in the lowest protein improvement. The final protein levels for all other fungal strains were relatively similar, at 40-41% in hexane extracted canola meal and 43-45% protein in cold pressed canola meal.

### Residual Sugars

Residual sugars represent the combined levels of arabinose, galactose, glucose, raffinose, stachyose, and sucrose. Between 50-95% of sugars present in the HE and CP meals were utilized by the fungi during incubation, resulting in residual sugar levels of 0.9-8.4% w/w. A. pullulans (NRRL-Y-2311-1) and F. venenatum exhibited the lowest residual sugar levels on both substrates, while M. circinelloides and T. reesei had the highest final levels of residual sugars. In the case of T. reesei, the higher than optimal final pH levels might explain why sugars were not more completely consumed. Table 3 shows the specific concentrations of raffinose and stachyose for SSF for the individual fungi.

TABLE 3
Reduction of raffinose and stachyose during solid
state fungal incubation
Solid State Incubation

| | HE | | CP | |
|---|---|---|---|---|
| | Stachyose | Raffinose | Stachyose | Raffinose |
| A. pullulans (58522) | 0.00 | 1.31 | 0.00 | 1.11 |
| A. pullulans (42023) | 0.00 | 1.41 | 0.00 | 1.28 |
| A. pullulans (Y- | 1.26 | 0.00 | 2.09 | 0.00 |
| 2311-1) | | | | |
| P. kudriavzeii | 1.45 | 1.65 | 0.53 | 1.86 |
| T. reesei | 0.00 | 1.45 | 0.51 | 1.33 |
| F. venenatum | 0.00 | 0.46 | 0.00 | 0.00 |
| M. circinelloides | 1.87 | 1.43 | 1.44 | 1.36 |

Raw canola meal: HE, stachyose 5.69 and raffinose 2.86; CP, stachyose 5.57 and raffinose 2.37.

Table 4 lists glucosinolate (GLS) and fiber (ADF and NDF) levels for the raw HE and CP canola meals, and for the meals following incubation (un-inoculated control vs. the various fungal cultures).

TABLE 4
Reduction of total glucosinolates and fiber during solid-state fungal incubation

| | Glucosinolates | | | | Fiber | | | |
|---|---|---|---|---|---|---|---|---|
| | Hexane Extracted | | Cold Pressed | | Hexane Extracted | | Cold Pressed | |
| | Total GLS | Reduction | Total GLS | Reduction | ADF | NDF | ADF | NDF |
| Fungal Culture | (μM/g) | (%)^{a} | (μM/g) | (%)^{a} | (%) | (%) | (%) | (%) |
| Raw Meal | 42.8 ± 1.3 | — | 60.6 ± 2.5 | — | 19.9 ± 0.2 | 23.1 ± 0.3 | 11.5 ± 0.5 | 15.0 ± 0.3 |
| Process Control | 14.8 ± 1.2 | 65.5 ± 2.7 | 29.9 ± 0.8 | 50.7 ± 1.3 | 17.2 ± 0.5 | 20.4 ± 0.6 | 9.7 ± 0.4 | 11.9 ± 0.6 |
| A. pullulans | 4.5 ± 1.0 | 89.4 ± 2.4 | 3.2 ± 2.5 | 94.8 ± 4.1 | 17.7 ± 1.3 | 21.3 ± 0.7 | 10.2 ± 1.0 | 12.9 ± 0.2 |
| (NRRL-58522) | | | | | | | | |
| A. pullulans | 2.6 ± 1.5 | 94.0 ± 3.5 | 15.1 ± 14.1 | 83.4 ± 21.9 | 18.0 ± 0.6 | 20.7 ± 0.7 | 10.5 ± 0.8 | 14.1 ± 0.9 |
| (NRRL-42023) | | | | | | | | |
| A. pullulans | 3.9 ± 1.0 | 90.9 ± 2.2 | 10.7 ± 2.7 | 82.4 ± 4.4 | 22.4 ± 0.7 | 25.8 ± 0.7 | 12.4 ± 0.7 | 15.4 ± 0.2 |
| (NRRL-Y-2311-1) | | | | | | | | |
| P. kudriavzevii | 13.3 ± 1.1 | 68.9 ± 2.5 | 28.7 ± 0.8 | 52.6 ± 1.3 | 18.7 ± 0.7 | 22.3 ± 0.6 | 10.6 ± 0.1 | 12.6 ± 0.5 |
| T. reesei | 0.4 ± 0.0 | 99.1 ± 0.1 | 1.0 ± 0.2 | 98.3 ± 0.3 | 19.1 ± 0.4 | 23.6 ± 0.8 | 11.0 ± 0.9 | 13.5 ± 0.7 |
| (NRRL-3653) | | | | | | | | |
| F. venenatum | 5.7 ± 1.6 | 86.7 ± 3.8 | 10.9 ± 1.7 | 82.1 ± 6.6 | 22.5 ± 0.7 | 28.9 ± 0.5 | 11.6 ± 1.2 | 14.6 ± 1.2 |
| (NRRL-26139) | | | | | | | | |
| M. circinelloides | 10.4 ± 1.0 | 75.8 ± 2.2 | 24.4 ± 2.7 | 58.1 ± 4.4 | 20.1 ± 0.8 | 23.4 ± 0.5 | 11.1 ± 0.8 | 13.3 ± 0.7 |

^{a}GLS reduction from raw canola meal

### Glucosinolates

GLS levels were reduced from 17.4 mg/g in raw hexane extracted meal to 6.0 mg/g after the thermal treatments in the control (autoclave sterilization and final drying), representing a 65.5% reduction. This was presumed due to volatilization of GLS breakdown products. Solid-state microbial conversion further reduced GLS content to 0.4-5.4 mg/g, representing a total reduction of 69-98%. Similarly, GLS levels in raw cold pressed meal were reduced from 24.7 mg/g to 12.2 mg/g due to the thermal steps of the conversion process (reduction of 50.7%). Again, solid state microbial conversion further reduced GLS content to 1.0-11.7 mg/g (total reduction of 53-96%).

T. reesei (NRRL-3653) exhibited the greatest reduction in glucosinolate levels, likely due to its robust capability for producing extracellular enzymes. Several of the A. pullulans strains were next most effective. Previous studies have shown that various microbes, including Aspergillus sp. and Rhizopus oligosporus, are able to degrade GLS and metabolize the resulting glucose and sulfur moieties. The complete degradation of glucosinolates was achieved after 60-96 h using solid-state fermentation with Aspergillus sp. As expected due to its minimal production of extracellular hydrolytic enzymes, P. kudriavzevii resulted in the least reduction in GLS.

### Fiber

Fiber levels (Table 4) actually increased during the fungal incubation process as a result of the “concentration effect” as sugars and glucosinolates were metabolized, along with the apparent lack of any substantial fiber hydrolysis due to cellulase activity.

Table 5 provides a summary of the dry matter yield and total amount of protein achieved in these trials.

TABLE 5
Dry matter yield and protein from solid
state incubation of canola meal

| | Hexane Extracted | | | Cold Pressed | | |
|---|---|---|---|---|---|---|
| | Dry | | | Dry | | |
| | Matter | | Total | Matter | | Total |
| | Yield | Protein | Protein | Yield | Protein | Protein |
| Fungal Culture | (%) | (%, dw) | (g) | (%) | (%, dw) | (g) |
| Control | 100 | 36.1 | 18.1 | 100 | 38.6 | 19.4 |
| A. pullulans | 95.2 | 41.0 | 19.5 | 92.0 | 45.2 | 20.8 |
| (NRRL-58522) | | | | | | |
| A. pullulans | 97.3 | 39.7 | 19.3 | 88.5 | 43.7 | 19.4 |
| (NRRL-42023) | | | | | | |
| A. pullulans | 91.0 | 41.3 | 18.8 | 90.6 | 44.6 | 20.2 |
| (NRRL-Y- | | | | | | |
| 2311-1) | | | | | | |
| P. kudriavzevii | 100 | 39.7 | 20.0 | 98.0 | 42.2 | 20.7 |
| T. reesei | 91.9 | 44.4 | 20.4 | 91.2 | 47.5 | 21.7 |
| (NRRL-3653) | | | | | | |
| F. venenatum | 92.5 | 40.2 | 18.6 | 91.5 | 44.4 | 20.3 |
| (NRRL-26139) | | | | | | |
| M. | 96.9 | 40.7 | 19.7 | 97.4 | 43.0 | 20.9 |
| circinelloides | | | | | | |

While the composition (protein, glucosinolate and fiber) of the microbially converted canola meal is important, the product yield (especially protein) is also important. The dry matter yield was calculated by dividing the final dry weight following incubation Based on dry matter yield and protein concentration, one can calculate the total protein content in the product, and on this basis, T. reesei performed the best for both HE and CP canola meal. M. circinelloides was the second best, while P. kudriavzevii was next, due to the high yield. Several of the filamentous fungi that produced relatively high protein levels actually yielded less total protein due to their reduced dry matter yields.

In summary, Solid-state incubation conditions were used to enhance filamentous growth of the fungi. Flask trials were performed using 50% moisture content hexane extracted or cold pressed canola meal, with incubation for 168 h at 30° C. On hexane extracted canola meal Trichoderma reesei (NRRL-3653) achieved the greatest increase in protein content (23%), while having the lowest residual levels of sugar (8% w/w) and glucosinolates (0.4 mg/g). On cold pressed canola meal Trichoderma reesei (NRRL-3653), A. pullulans (NRRL-58522), and A. pullulans (NRRL-Y-2311-1) resulted in the greatest improvement in protein content (22.9, 16.9 and 15.4%, respectively), while reducing total glucosinolate content to 1.0, 1.2 and 4.3 mg/g, respectively. Fiber levels increased due to the concentration effect of removing oligosaccharides and glucosinolates.

### Example 2

### Submerged Trials

Seven fungal strains were grown on HE v CP canola meal using a submerged incubation process. The fungi were tested both on raw (non-saccharified) and saccharified meal slurries using commercial cellulases to enhance fiber breakdown. These trials were carried out in shake flasks, where mixing and mass transfer may be limiting factors.

### Total Protein

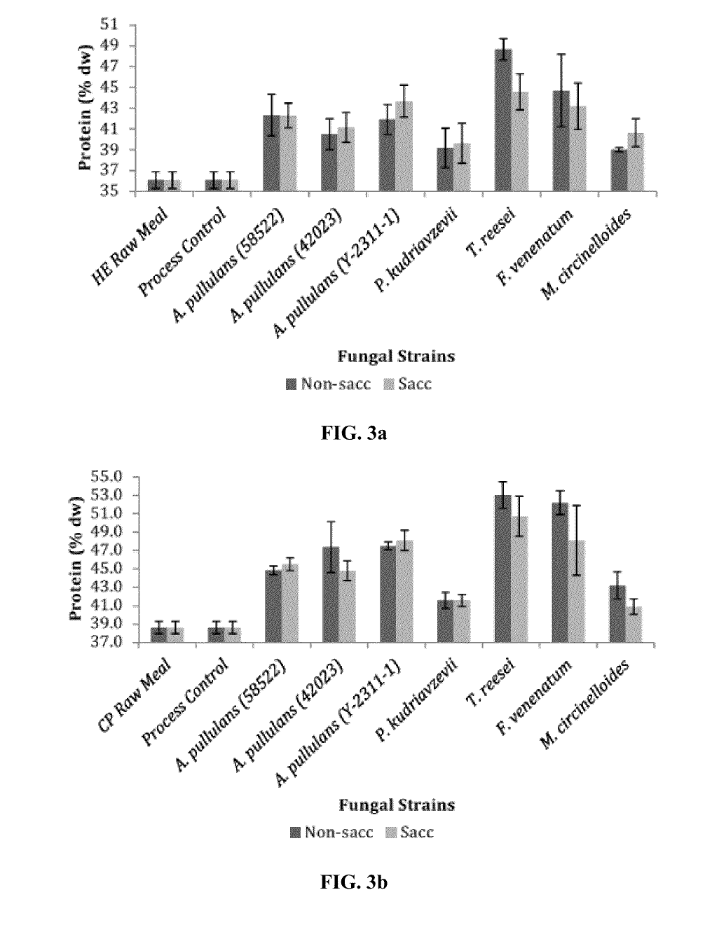

FIGS. 3a and 3b present the maximum protein levels in HE and CP canola meals, respectively, for raw meal and un-inoculated controls versus the various fungi, both under non-saccharified and saccharified conditions. As expected, protein levels for the un-inoculated controls were similar to the raw meals. In HE meal, protein levels increased from 36.1% in the raw meal to 39.0-48.7% after the fungal conversion process (relative improvements of ˜8.0-34.9%) (FIG. 3a). The M. circinelloides trial was the only one in which an enzymatic hydrolysis step prior to inoculation proved beneficial. In the case of T. reesei, the non-saccharified trial resulted in higher protein titers. It was anticipated that saccharification would have a significant positive effect on fiber hydrolysis, and subsequently protein levels. As such, this was an unexpected result. While not being bound by theory, it could be that canola fibers require pre-treatment to increase susceptibility to enzymatic hydrolysis.

In the CP canola meal (FIG. 3b) the protein level in the un-inoculated control was 38.6%, and rose to 40.9-53.0% after microbial conversion, representing relative improvements of ˜6.0-37.3%. CP canola meal was ˜3% higher in protein than HE meal, and following incubation, protein levels were ˜2-8% higher in CP canola meal trials compared to HE meal for each pair of fungi. While not being bound by theory, HE seems to be a more effective method of removing oil from canola seed, however, this process applies significantly higher levels of heat, which may denature or degrade some proteins. It was observed that the enzymatic hydrolysis step prior to inoculation did not significantly affect protein levels for all the fungi tested. The, for un-pretreated canola meal, it seems that there was no benefit to adding cellulolytic enzymes.

T. reesei achieved the highest protein levels for both substrates, while P. kudriavzevii exhibited the lowest protein enhancement. The final protein levels for all other fungal strains was relatively similar, at 40-45% in HE canola meal and 43-53% protein in CP canola meal.

### Residual Sugars

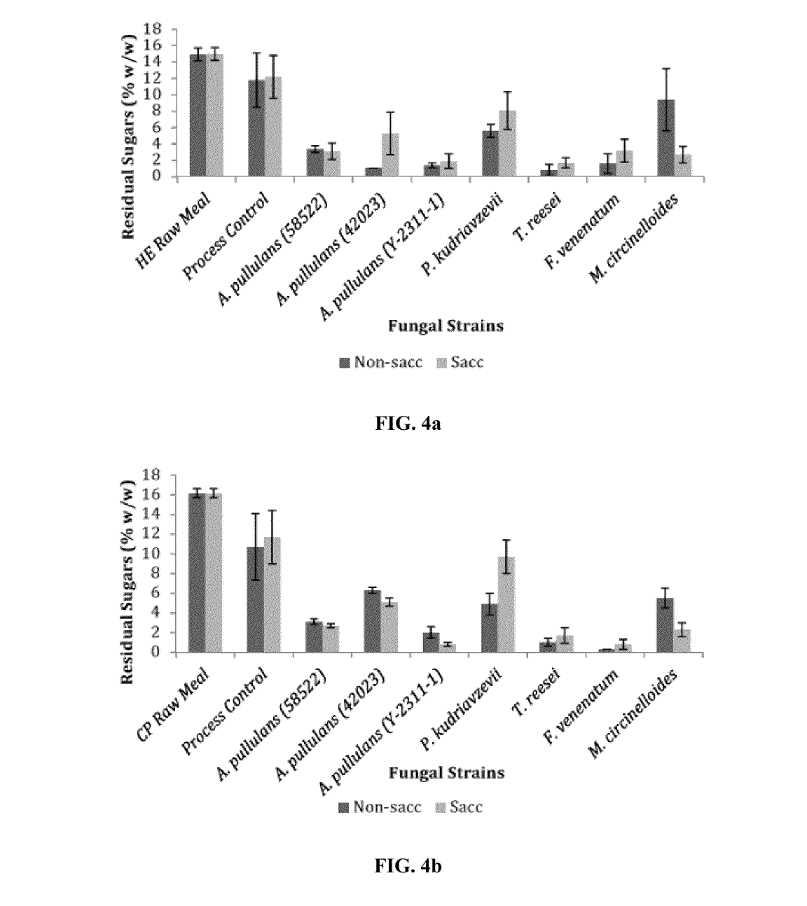

Arabinose, galactose, glucose, raffinose, stachyose, and sucrose were measured throughout incubation via HPLC. For simplicity, final levels of these sugars were combined and are presented as residual sugars in FIGS. 4a and 4b for HE and CP canola meals, respectively. The total residual sugar concentrations decreased slightly (2.7-5.5%) from the raw meals compare to the process controls. While not being bound by theory, autoclaving the 10% SLR canola slurries seem to have hydrolyzed oligosaccharides, raffinose and stachyose, thus reducing their concentrations. Stachyose and raffinose concentrations are shown in Tables 6 and 7.

TABLE 6
Reduction of raffinose and stachyose during submerged
fungal incubation (saccharification)
Submerged Incubation
Saccharification

| | HE | | CP | |
|---|---|---|---|---|
| | Stachyose | Raffinose | Stachyose | Raffinose |
| A. pullulans (58522) | 1.22 | 0.71 | 0.00 | 1.18 |
| A. pullulans (42023) | 2.12 | 1.23 | 2.06 | 0.37 |
| A. pullulans (Y- | 0.00 | 0.44 | 0.60 | 0.01 |
| 2311-1) | | | | |
| P. kudriavzeii | 1.89 | 2.39 | 1.96 | 1.51 |
| T. reesei | 0.52 | 1.06 | 1.04 | 0.58 |
| F. venenatum | 0.56 | 1.28 | 0.76 | 0.58 |
| M. circinelloides | 0.00 | 1.46 | 0.00 | 0.43 |

TABLE 7
Reduction of raffinose and stachyose during submerged
fungal incubation (non-saccharification)
Submerged Incubation
Non Saccharification

| | HE | | CP | |
|---|---|---|---|---|
| | Stachyose | Raffinose | Stachyose | Raffinose |
| A. pullulans (58522) | 0.09 | 0.41 | 0.37 | 0.08 |
| A. pullulans (42023) | 0.00 | 0.32 | 2.88 | 0.00 |
| A. pullulans (Y- | 0.00 | 0.00 | 0.00 | 0.00 |
| 2311-1) | | | | |
| P. kudriavzeii | 1.37 | 2.11 | 0.24 | 1.20 |
| T. reesei | 0.09 | 0.42 | 0.00 | 0.84 |
| F. venenatum | 0.52 | 1.06 | 0.00 | 0.14 |
| M. circinelloides | 1.44 | 1.49 | 1.18 | 1.45 |

In non-saccharified HE meal (FIG. 4a) between 37.0-94.6 of sugars present were used by the fungi during incubation, resulting in residual sugar levels of 0.8-9.4%. Similarly, 39.0-88.6% of sugars present in the saccharified HE meal was utilized by the fungi, resulting in residual sugars levels of 1.7-9.1%. T. reesei exhibited the lowest residual sugar levels on both non-saccharified and saccharified HE meals, while M. circinelloides and P. kudriavzevii had the highest final levels in non-saccharified and saccharified trials, respectively. M. circinelloides did show a benefit from saccharification, showing a significant drop in residual sugars from 9.4 to 2.75 w/w when compared to non-saccharification.

In non-saccharified CP meal (FIG. 4b) between 61.0-98.1% of sugars present were metabolized by the fungi during incubation, decreasing residual sugar levels to 0.3-6.3%. Similarly, 40.0-95.0% of sugars present in saccharified CP meal were metabolized by the fungi during incubation, decreasing residual sugar levels to 0.8-9.7%. F. venenatum and T. reesei exhibited the lowest residual sugar levels on both non-saccharified and saccharified COP meal, while A. pullulans (NRRL-42023) and P. kudriavzevii had the highest final levels in non-saccharified material, respectively. Saccharification significantly reduced residual sugars in trials with M. circinelloides and A. pullulans (NRRL-42023) when compared to non-saccharification trials.

### Glucosinolates

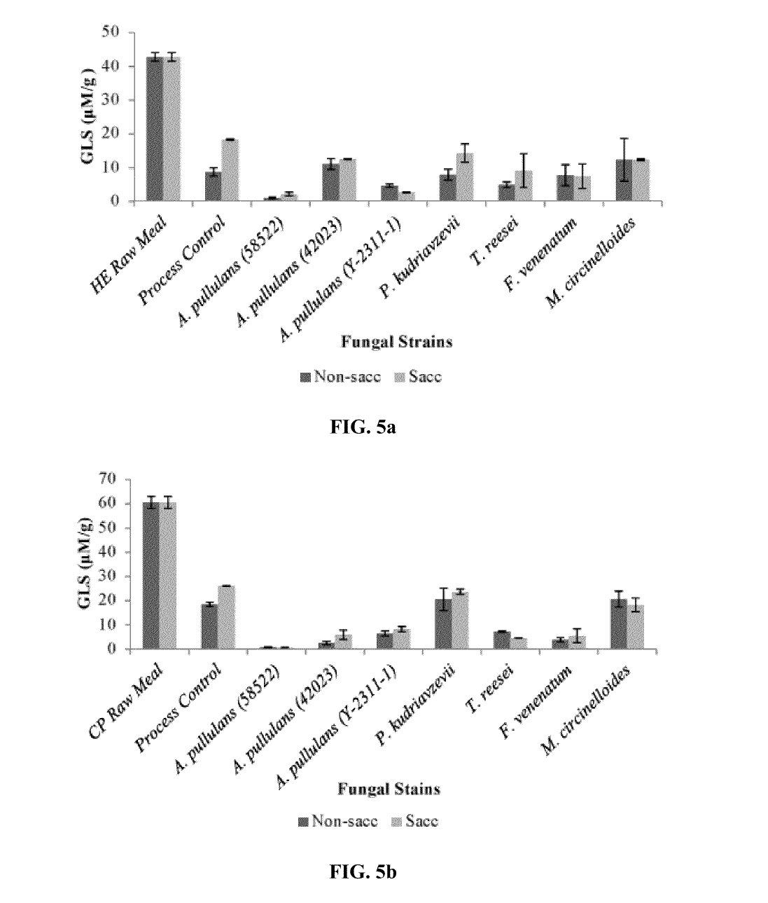

FIGS. 5a and 5b show GLS levels for the HE and CP canola meal trials, respectively. GLS levels were reduced form 42.8 μM/g in raw HE meal to 8.7 μM/g (non-saccharified) and 18.3 μM/g (saccharified) in the un-inoculated process controls. This represents 79.6 and 57.2% reductions, respectively, and, while not to be bound by theory, was presumed due to the conversion of some of the GLS into volatile breakdown products. Submerged microbial conversion further reduced GLS content to 1.0-14.4 μM/g, representing a total reduction of 66.5-97.8%.

GLS levels in raw CP meal (60.6 μM/g) were higher than in HE meal (42.8 μM/g) since the former does not include high temperature de-solventing step which can eliminate GLS. Treatment of the CP meal with the autoclaving and drying steps in the process control reduced GLS levels to 18.6 and 26.2 μM/g, respectively in non-saccharified and saccharified trials (reduction of 69.4 and 56.8), respectively. Again, submerged microbial conversion further reduced GLS content to 0.7-23.7 μM/g (total reduction of 60.8-98.9%).

Overall, A. pullulans (NRRL-58522) caused the greatest reduction in GLS levels in both HE and CP canola meals (ranging from 94.5-98.9%). A. pullulans (NRRL-Y-2311-1) was also very effective in reducing GLS concentrations (ranging from 86.3-93.7%), followed by F. venenatum (81.8-93.5%) and T. reesei (78.7-92.2%). Further, P. kudriavzevii and M. circinelloides resulted in the least reduction in GLS.

### Fiber

Table 8 provides the ADF and NDF fiber levels of raw, process control, and treated canola meals.

TABLE 8
Fiber reduction of non-saccharified and saccharified canola meal during submerged fungal incubation

| | Hexane Extracted | | | | Cold Pressed | | | |
|---|---|---|---|---|---|---|---|---|
| | Non-saccharified | | Saccharified | | Non-saccharified | | Saccharified | |
| Fungal Culture | ADF (%) | NDF (%) | ADF (%) | NDF (%) | ADF (%) | NDF (%) | ADF (%) | NDF (%) |
| Raw Meal | 19.9 ± 0.2 | 23.1 ± 0.3 | 19.9 ± 0.2 | 23.1 ± 0.3 | 11.5 ± 0.5 | 15.0 ± 0.3 | 11.5 ± 0.5 | 15.0 ± 0.3 |
| Process Control | 18.7 ± 0.3 | 22.0 ± 0.8 | 23.0 ± 1.1 | 29.0 ± 1.8 | 9.5 ± 0.6 | 12.4 ± 0.8 | 14.8 ± 1.4 | 16.1 ± 1.6 |
| A. pullulans | 22.0 ± 1.6^{b} | 29.1 ± 0.8^{b} | 20.6 ± 2.4 | 25.2 ± 4.6 | 12.1 ± 1.0 | 16.8 ± 0.7^{b} | 11.6 ± 1.1 | 15.9 ± 0.6 |
| (NRRL-58522) | | | | | | | | |
| A. pullulans | 20.4 ± 1.5 | 24.3 ± 1.3 | 19.4 ± 2.2 | 22.6 ± 0.5 | 12.4 ± 0.4 | 16.9 ± 0.4^{b} | 11.2 ± 1.1 | 15.1 ± 0.3 |
| (NRRL-42023) | | | | | | | | |
| A. pullulans | 22.3 ± 0.9^{b} | 24.5 ± 0.7^{b} | 21.2 ± 1.4 | 24.0 ± 0.9 | 13.6 ± 2.1 | 14.8 ± 2.4 | 11.0 ± 0.9 | 12.6 ± 1.0^{a} |
| (NRRL-Y-2311-1) | | | | | | | | |
| P. kudriavzevii | 19.7 ± 1.6 | 23.1 ± 1.9 | 18.6 ± 0.3^{a} | 22.7 ± 1.9 | 11.0 ± 0.4 | 13.5 ± 0.3^{a} | 10.4 ± 0.4^{a} | 12.4 ± 0.5^{a} |
| T. reesei | 19.9 ± 3.1 | 22.5 ± 4.0 | 19.8 ± 0.8 | 26.4 ± 3.3 | 7.6 ± 0.8^{a} | 10.1 ± 0.6^{a} | 8.1 ± 1.2^{a} | 10.8 ± 2.0^{a} |
| (NRRL-3653) | | | | | | | | |
| F. venenatum | 21.3 ± 2.4 | 26.7 ± 2.6^{b} | 20.8 ± 2.2 | 26.9 ± 0.7^{b} | 7.6 ± 0.9^{a} | 10.7 ± 1.2^{a} | 10.7 ± 2.5 | 12.9 ± 3.0 |
| (NRRL-26139) | | | | | | | | |
| M. circinelloides | 21.0 ± 0.5^{b} | 25.9 ± 1.2 | 19.6 ± 1.2 | 22.6 ± 1.0 | 10.2 ± 0.9 | 12.8 ± 0.9^{a} | 10.8 ± 0.6 | 15.6 ± 1.3 |

^{a}Indicates fiber level was statistically lower than raw meal
^{b}Indicates fiber level was statistically higher than raw meal

In general, most fiber levels were statistically similar to the raw meal, indicating that the conversion process had minimal effects on fiber levels. The only trial to show a statistically significant reduction in ADF in HE meal was P. kudriavzevii, while trials with A. pullulans (NRRL-Y-2311-1), P. kidriavzevii, T. reesei, F. venenatum, and M. circinelloides all statistically reduced ADF and/or NDF fiber levels in CP canola meal (Table 8). Thus, the cellulose producing fungi were effective in hydrolyzing fiber in CP canola meal, however, did not show similar results in HE canola meal.

In some cases the conversion process resulted in a concentration of fibers, caused by the removal of sugars and GLS. Trials with A. pullulans (58522), A. pullulans (NRRL-Y-2311-1), F. venenatum, and M. circinelloides all increased fiber levels in HE canola meal, while A. pullulans (58522) and A. pullulans (42023) treatments both increased ADF and/or NDF fiber levels in CP canola meals (Table 8).

Submerged incubation with various fungal strains improved the nutritional content of canola meal. T. reesei (NRRL-3653), F. venenatum (NRRL-26139), and A. pullulans (NRRL-Y-2311-1) resulted in the greatest improvement in protein content in HE canola meal (34.8, 23.8, and 21.0%, respectively), while reducing total GLS and residual sugar content by 82.6-93.7% and 89.3-94.6%. In trials with CP canola meal, the same three fungi increased protein levels to the greatest extent (37.3, 35.2, and 24.6%, respectively), while reducing total GLS and residual sugar content by 89.3-93.5% and 93.8-98.1%.

### Example 3

### Pretreatment Trials

Three fugal strains were grown on pretreated and non-pretreated HE versus CP canola meal using a submerged incubation process. These trials were done in shake flasks, where mixing and mass transfer are limiting factors.

### Total Protein

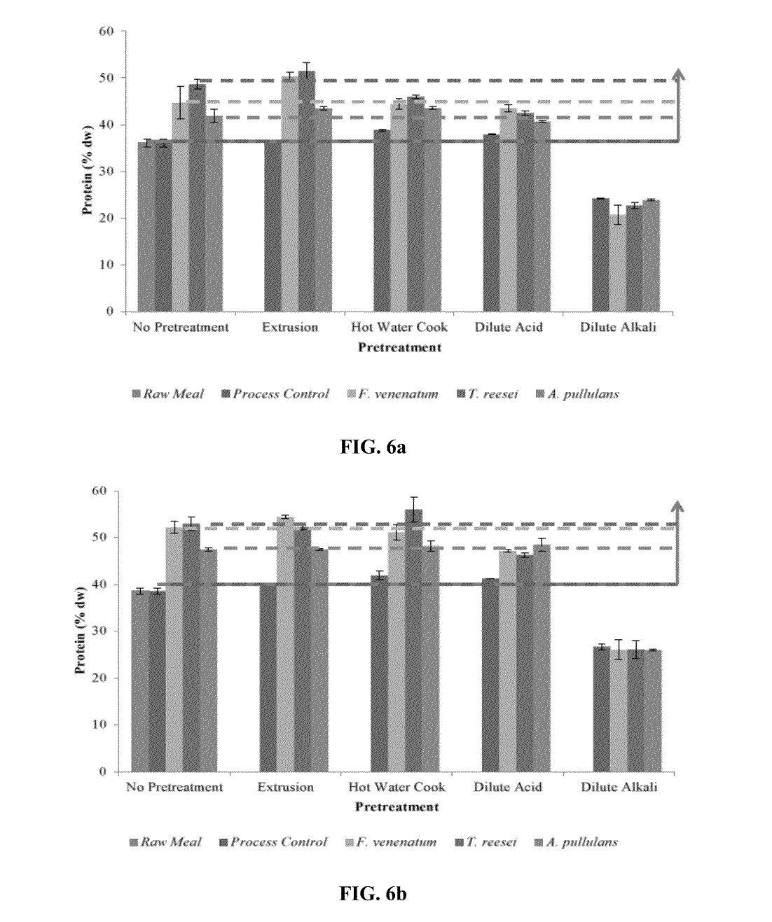

FIGS. 6a and 6b represent the maximum protein levels achieved during the various treatments with HE and CP canola meal, respectively. The initial protein level of the raw, un-pretreated meals is provided, along with process control samples, which were processed identically to the other treatments within each series, except that they were not inoculated with fungi. Hence the process controls represent the effects of the pretreatment, autoclaving, and drying steps.

As shown in FIG. 6a, extrusion pretreatment by itself did not affect protein levels, and very slight increases were observed for the process controls in the hot water cook and dilute acid pretreatments. However, the dilute alkali pretreatment resulted in a large reduction in true protein, dropping levels from ˜36% to ˜25% in the respective process controls. While not being bound by theory, the alkali treatment may have cleaved ammonia groups from amino acids. Dilute alkali pretreatment may also cause other adverse effects on the nutritional quality of livestock meals.

The dilute alkali pretreatment used a 4% ammonia concentration, and because the final meal following pretreatment and incubation contained high levels of ammonia, all these samples were assayed for NPN levels. The NPN value was then subtracted from the LECO nitrogen analyzer data so the true protein content could be calculated. For example, in the case of HE canola meal, the LECO N content (12.7%) was subtracted from the NPN content (8.8%), resulting in a nitrogen level of 3.9%, which converts into a protein content of 24.17%.

From the submerged study above, seven fungal strains incubated on non-pretreated HE canola meal showed that protein levels increased from 36.1% in raw meal to 41.9-48.7% after incubation, with the three best fungi demonstrating the following order: T. reesei>F. venenatum>A. pullulans. This represented relative improvements of ˜16.1-34.8%. Similar results were observed here with the un-pretreated, extrusion pretreated, and hot water cook pretreated HE canola meal, with T. reesei achieving the highest protein level, although it was not statistically different from F. venenatum.

Overall, extrusion was the most effective pretreatment for HE canola meal, achieving protein levels of 51.5, 50.4, and 43.5 for T. reesei, F. venenatum, and A. pullulans, respectively. Hot water cook and dilute acid pretreatments were relatively similar to the un-pretreated control, however, dilute alkali pretreatment reduced protein levels to 20.7-24.2, representing a protein loss of ˜33.0-42.7%. Dilute alkali was also the only pretreatment in which subsequent fungal incubation did not increase the protein content compared to the process control. While not being bound by theory, the enhanced degradation of fiber from dilute alkali pretreatment may have produced inhibitory compounds (e.g., furfural and hydroxyfurfural) that could have prevented fungal single-cell protein production.

FIG. 6b shows similar data for pretreatments of the CP canola meal. Again, extrusion pretreatment by itself did not affect protein level, while the process controls in the hot water cook and dilute acid pretreatments showed very slight gains in protein. The dilute alkali pretreatment one again resulted in a significant loss of protein.

From the submerged study above, fungal performance was evaluated on non-pretreated CP canola (38.6%), and protein levels increase to 47.5-53.0% with the same three fungi in the same order, representing relative improvements of ˜23.0-37.3%. FIG. 6b shows similar performance improvements by the three fungi on the same in-pretreated feedstock. For the un-pretreated extrusion, and hot water pretreated samples, T. reesei and F. venenatum performed better than A. pullulans. However, unlike the trials with HE canola meal where extrusion was the best pretreatment (FIG. 6a), both hot water cook and extrusion resulted in similar performance, which was not much different than the un-pretreated control. Dilute acid pretreatment slightly reduced protein levels, while dilute alkali pretreated caused a significant reduction in protein levels.

In summary, T. reesei achieved the highest protein levels for both substrates, while A. pullulans exhibited to lowest protein enhancements. Extrusion was of HE was the only pretreatment to show a consistent boost in protein levels following incubation with the three fungi. Dilute alkali pretreatment resulted in a significant loss of protein in all cases. Maximal protein levels for the other pretreatments were similar to the un-pretreated control.

### Residual Sugars

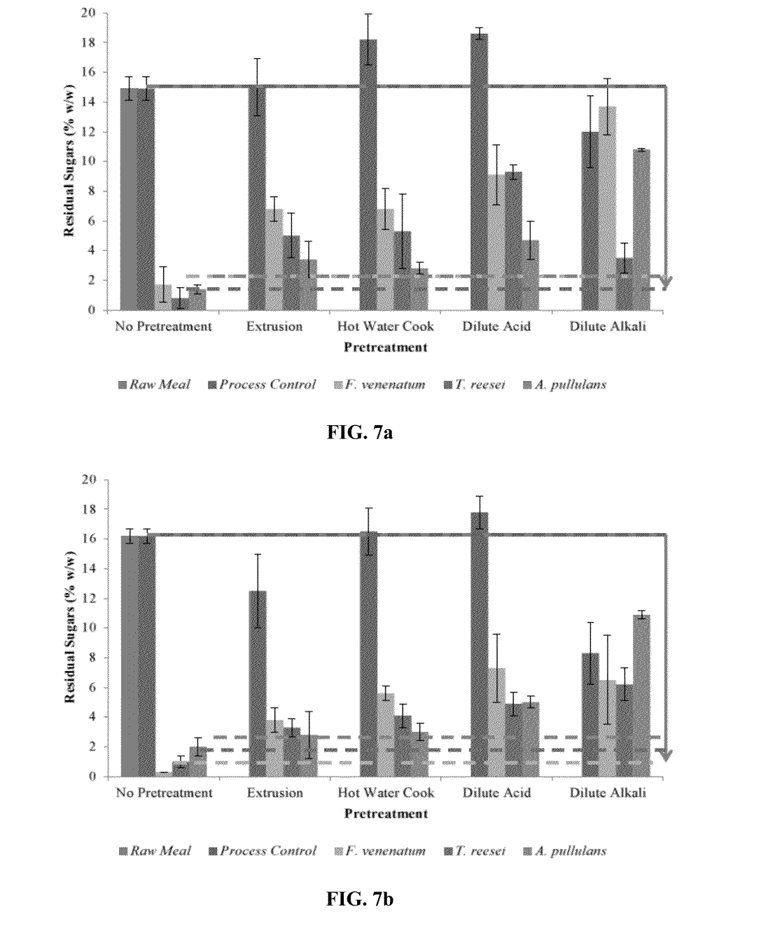

FIG. 7a provides the total residual sugar levels following pretreatment and fungal incubation of HE canola meal. The residual sugar content represent combined levels of arabinose, galactose, glucose, raffinose, stachyose, and sucrose. The un-inoculated process controls showed that the hot water cook (18.2%) and dilute acid pretreatments (18.6%) increased residual sugar levels compared to the un-pretreated control (14.9%). These results suggest that these pretreatments hydrolyzed oligosaccharides, such as pectin, into shorter chain carbohydrates, including arabinose, galactose, glucose, xylose, and mannose. Extrusion pretreatment did not affect residual sugar levels, while dilute alkali pretreatment resulted in a decrease in residual sugar content at 12%. Monosaccharides, such as glucose, galactose, and mannose, are rapidly destroyed by the hot aqueous alkali solution used with dilute alkali pretreatment. Stachyose and raffinose concentrations are shown in Tables 9-12.

TABLE 9
Change in raffinose and stachyose concentration during
submerged fungal incubation (pretreatment-hot water cook)
Pretreatment with Submerged Incubation
Hot Water Cook

| | HE | | CP | |
|---|---|---|---|---|
| | Stachyose | Raffinose | Stachyose | Raffinose |
| A. pullulans (Y- | 0.00 | 0.48 | 0.17 | 0.49 |
| 2311-1) | | | | |
| F. venenatum | 2.62 | 1.69 | 2.79 | 0.00 |
| T. reesei | 2.08 | 0.31 | 1.49 | 0.22 |

TABLE 10
Change in raffinose and stachyose concentration during
submerged fungal incubation (pretreatment-extrusion)
Pretreatment with Submerged Incubation
Extrusion

| | HE | | CP | |
|---|---|---|---|---|
| | Stachyose | Raffinose | Stachyose | Raffinose |
| A. pullulans (Y- | 0.30 | 0.18 | 0.13 | 0.49 |
| 2311-1) | | | | |
| F. venenatum | 2.42 | 0.00 | 0.94 | 0.24 |
| T. reesei | 1.52 | 0.31 | 0.31 | 0.24 |

TABLE 11
Change in raffinose and stachyose concentration during
submerged fungal incubation (pretreatment-dilute acid)
Pretreatment with Submerged Incubation
Dilute Acid

| | HE | | CP | |
|---|---|---|---|---|
| | Stachyose | Raffinose | Stachyose | Raffinose |
| A. pullulans (Y- | 0.36 | 0.90 | 0.64 | 0.69 |
| 2311-1) | | | | |
| F. venenatum | 1.61 | 1.46 | 1.62 | 0.43 |
| T. reesei | 0.63 | 1.20 | 0.00 | 0.69 |

TABLE 12
Change in raffinose and stachyose concentration during
submerged fungal incubation (pretreatment-dilute alkali)
Pretreatment with Submerged Incubation
Dilute Alkali

| | HE | | CP | |
|---|---|---|---|---|
| | Stachyose | Raffinose | Stachyose | Raffinose |
| A. pullulans (Y- | 0.00 | 3.65 | 0.00 | 3.36 |
| 2311-1) | | | | |
| F. venenatum | 0.00 | 3.24 | 0.00 | 2.32 |
| T. reesei | 0.60 | 0.77 | 1.05 | 2.00 |

Following fungal incubation, the non-pretreated HE canola meal samples shows the lowest residual sugar content, ranging form 0.8, 1.4, and 1.7/% with T. reesei, A. pullulans, and F. venenatum, respectively. For the pretreated samples, A. pullulans depleted sugar levels to the greatest extent, with the exception of the dilute alkali trial. In comparing the four pretreatments, extrusion and hot water cook had the lowest residual sugar tiers, while dilute alkali pretreatment had the highest.

FIG. 7b provides the total residual sugar levels following pretreatment and incubation of CP canola meal. In the un-inoculated process controls, the dilute acid pretreatment showed a slightly higher sugar level (17.8%) compared to the un-pretreated control (16.2%), while the hot water cook pretreatment was similar. Both the extrusion (12.5%) and dilute alkali (8.3%) pretreatments showed decreased residual sugar contents. In general, the remaining comparisons were similar to those noted in FIG. 7a for HE canola meal. The non-pretreated CP canola meal showed the lowest residual sugar levels after fungal incubation, while dilute alkali pretreatment was the highest.

### Fiber

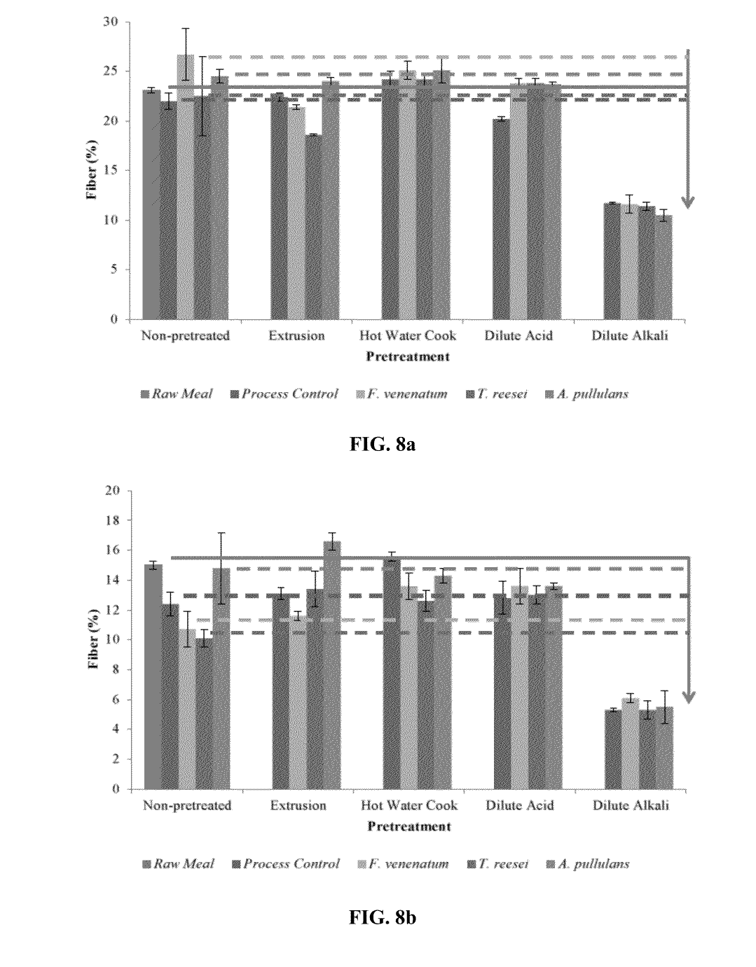

FIGS. 8a and 8b provide the NDF levels following pretreatment and incubation of HE and CP canola meals, respectively. Dilute alkali was the most effective pretreatment in reducing NDF levels, achieving 54.5% and 64.7% reductions in the un-inoculated process controls compared to the raw HE and CP canola meals, respectively. The other less obvious trend was that the hot water cook pretreatment resulted in a slight increase in NDF levels, and while not being bound by theory, this may result from solubilizing other components (e.g., oligosaccharides), and thus, “concentrating” NDF.

There were no strong trends regarding the effects of the fungal incubation process, but trials with T. reesei and F. venenatum typically had lower residual fiber levels than A. pullulans. In some instances (FIGS. 8a and 8b), fiber levels actually increased during the fungal incubation process as a result of the “concentration effect” as sugars and GLS were metabolized and protein levels increased (see FIGS. 6a and 6b).

### Glucosinolates

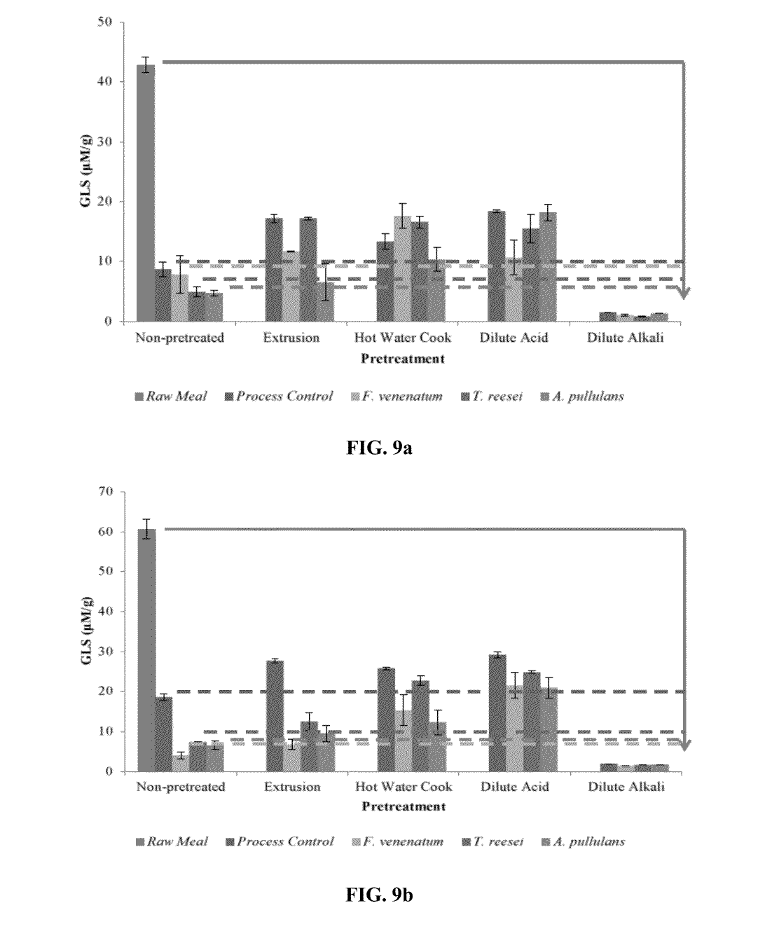

FIGS. 9a and 9b show GLS levels following pretreatment and fungal incubation of HE and CP canola meals, respectively. The un-inoculated process controls for the non-pretreated feedstocks showed that the autoclave and final drying steps of the process degraded and/or volatilized significant amounts of GLS. In HE meal, GLS levels were reduced form 42.8 μM/g in raw meal to 8.7 μM/g after these treatments, representing a 79.9% reduction. Similarly, GLS levels in raw CP meal were reduced from 60.6 μM/g to 18.6 μM/g due to the thermal steps of the conversion process (i.e., a reduction of 69.3%). CP processing uses significantly lower processing temperatures than HE processing, which explains the higher starting GLS content in CP versus HE canola meals.

Dilute alkali was the only pretreatment that reduced total GLS content to lower levels than non-pretreatment meals in un-inoculated process controls of HE and CP canola meal (dilute alkali<non-pretreatment<hot cook<extrusion<dilute acid). FIGS. 9a and 9b). Surprisingly, GLS levels in the process controls in the extrusion, hot water cook, and dilute acid pretreatment were higher that the corresponding non-pretreatment trials.

Submerged microbial conversion further reduced GLS content in almost all cases to below that of the corresponding un-inoculated process control, however, there was not consistent pattern in the most effective fungi.

Pretreatment, followed by submerged fungal incubation, improved the nutritional content of canola meal. Extrusion and T. reesei (NRRL-3653) incubation resulted in the greatest overall improvement to HE canola meal, increasing protein to 51.5% and reducing NDF, GLS, and residual sugars to 18.6%, 17.2 μM/g, and 5% w/w, respectively. Extrusion and F. venenatum (NRRL-26139) incubation performed to best with CP canola meal, resulting in 54.4% protein while reducing NDF, GLS, and residual sugars to 11.6%, 6.7 μM/g, and 3.8 w/w, respectively.

Although the invention has been described with reference to the above examples, it will be understood that modifications and variations are encompassed within the spirit and scope of the invention. Accordingly, the invention is limited only by the following claims.

We claim herein:

1. A method of producing a protein concentrate from canola using fermentation comprising:
contacting canola containing substrate with a microbe selected from the group consisting of Trichoderma reesei and Fusarium venenatum;
incubating the contacted substrate for sufficient time, and at a pH of between about 4.0 to about 6.0, to convert the carbohydrates contained in said substrate into protein to form a protein concentrate; and
isolating the resulting protein concentrate,
wherein the resulting protein concentrate exhibits a reduction in glucosinolate concentration of between about 69% to about 98%.

2. The method of claim 1, wherein fermentation is solid state fermentation (SSF) or submerged fermentation (SMF) or a hybrid of SSF and SMF.

3. The method of claim 1, wherein the microbe is Trichoderma reesei.

4. The method of claim 2, wherein the fermentation is SSF.

5. The method of claim 4, wherein the moisture content of the substrate is between about 40% to about 60%.

6. The method of claim 1, where the protein concentration of the substrate increase between about 15% to about 23%.

7. The method of claim 1, wherein the substrate is thermally treated prior to contact with the microbe.

8. The method of claim 1, wherein the substrate is hexane extracted (HE) or cold pressed (CP).

9. The method of claim 1, wherein incubation is carried out without perturbing the substrate.

10. A protein concentrate produced by the method of claim 1.

11. A method of producing a non-animal based protein concentrate comprising:
pelletizing a substantially dry substrate selected from the group consisting of cereal grains, bran, sawdust, peat, oil-seed materials, wood chips, and combinations thereof;
passing moisture through said pelletized substrate;
inoculating said moisturized pellets by fogging atomized inoculant onto said moisturized pellets, wherein said inoculant comprises at least one microbe;
incubating the inoculated pellets at a suitable temperature under solid state fermentation conditions in a first chamber;
transferring said incubated pellets to one or more second chambers;
drying and milling said transferred pellets; and
recovering the resulting protein concentrate comprising the at least one microbe.

12. The method of claim 11, wherein the at least one microbe is selected from the group consisting of Aureobasidium pullulans, Sclerotium glucanicum, Sphingomonas paucimobilis, Ralstonia eutropha, Rhodospirillum rubrum, Kluyveromyces and Pichia spp, Trichoderma reesei, Pleurotus ostreatus, Rhizopus spp, and combinations thereof.

13. The method of claim 11, further comprising exposing said pellets to UV light prior to inoculating said moisturized pellets.

14. A protein concentrate derived from the method of claim 11.

15. A method of producing a protein concentrate from canola using fermentation comprising:
contacting canola containing, liquid substrate with a microbe selected from the group consisting of Trichoderma reesei and Fusarium venenatum;
incubating the contacted substrate for sufficient time, and at a pH of between about 4.0 to about 6.0, to convert the carbohydrates contained in said substrate into protein to form a protein concentrate, wherein incubation is carried out with perturbation of the substrate; and
isolating the resulting protein concentrate,
wherein the resulting protein concentrate exhibits a reduction in glucosinolate concentration of between about 69% to about 98%.

16. The method of claim 15, wherein the canola substrate comprises extruded canola meal.

17. The method of claim 16, wherein the canola meal is hexane extracted (HE) or cold pressed (CP).

18. The method of claim 15, wherein incubation is carried out for about 168 hours.

19. The method of claim 15, further comprising drying the resulting isolated protein concentrate.

20. A protein concentrate derived from the method of claim 15.
